(19) United States
(12) Patent Application Publication
(10) Pub. No.: US 2024/0139260 A1
(43) Pub. Date: May 2, 2024

(54) MICROBIAL STRAIN COMPOSITIONS

(71) Applicant: MICROBIAL DISCOVERY GROUP, LLC, Franklin, WI (US)

(72) Inventors: Michael R. King, Oak Creek, WI (US); Sona Son, Franklin, WI (US); Jessica Edward, Kenosha, WI (US); Kyle Leistikow, Franklin, WI (US)

(21) Appl. No.: 18/481,999

(22) Filed: Oct. 5, 2023

Related U.S. Application Data
(60) Provisional application No. 63/413,419, filed on Oct. 5, 2022.
(60) Provisional application No. 63/472,076, filed on Jun. 9, 2023.

(51) Int. Cl. A61K 35/742; C12N 1/20; C12R 1/07

(52) U.S. Cl. CPC A61K 35/742; C12N 1/205; C12R 2001/07

(57) ABSTRACT

The invention relates to microbials for use in improving the health of a human and animals. More particularly, the invention relates to isolated Bacillus strains, and strains having all of the identifying characteristics of these strains, for a use comprising the above-mentioned use, in particular for improving the gastrointestinal health of animals or humans.

### CROSS-REFERENCE TO RELATED APPLICATIONS

This application claims the benefit under 35 USC § 119(e) of U.S. Provisional Application Ser. No. 63/413,419, filed on Oct. 5, 2022 and U.S. Provisional Application Ser. No. 63/472,076, filed on Jun. 9, 2023, the entire disclosure of all of which are incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 20 kilobytes xml file named “395736.xml,” created on Nov. 20, 2023.

### FIELD OF THE DISCLOSURE

The invention relates to microbials for use in improving the health of humans and animals. More particularly, the invention relates to isolated Bacillus strains, and strains having all of the identifying characteristics of these strains, for a use comprising the above-mentioned use, in particular for improving the gastrointestinal health of animals or humans.

### BACKGROUND AND SUMMARY OF THE INVENTION

The present invention relates to probiotic compositions and methods for improving the health of animals or humans, particularly gastrointestinal health. The present invention also relates to such probiotic strains and methods for assessing their beneficial functional contributions to the animal or human ecosystem; such as digestive enzyme production, antimicrobial activity (E. coli, Salmonella, Campylobacter, Clostridium perfringens, Candida albicans, Candida auris, Enterococcus cecorum, Fusarium graminearium, and Aspergillus flavus), immunomodulation, antioxidant capacity, and quorum quenching capacity. While a “healthy intestinal microbiome” has yet to be precisely defined, it has been well-established over the years that diversity and balance of the microorganisms are crucial components. Beneficial bacteria, including the strains described in this application, are an important part of the gastrointestinal tract's environment because they provide animals and humans with bacteria that assist in establishment (or reestablishment) of a normal bacterial profile, they strengthen the immune system, they help to fight disease through antimicrobial metabolites and quorum quenching capacity (e.g., disease caused by the above-mentioned gram-negatives, gram-positives, yeasts, and molds), and they help to reduce inflammation by producing antioxidative enzymes.

An animal or a human's gastrointestinal tract is constantly challenged by large numbers of bacteria and viruses found in the environment. The gastrointestinal tract has a sophisticated system to counter these potential pathogens consisting of physical, chemical, and immunological lines of defense. Beneficial bacteria are an important part of this system. Pathogens, stress, metabolic upset, the use of antimicrobials, and other causes can upset the balance of intestinal bacteria, which may impair digestion and make an animal or a human more susceptible to disease.

Probiotics (i.e., also referred to in this application as direct-fed microbials) are products that contain live (viable) microorganisms (e.g., bacteria). Over time, many of the probiotic products previously considered useful for improving health have lost overall efficacy. Thus, additional microbial strains are needed that will improve animal and human health. Applicant has developed such probiotic compositions comprising Bacillus subtilis and Bacillus coagulans strains for improvement of animal health and human health.

Methods and compositions are provided for improving health in animals and humans. In various embodiments, the animal can be selected from the group consisting of a poultry species, a porcine species, a bovine species, an ovine species, an equine species, and a companion animal. In the embodiment where the animal is a poultry species, the poultry species can be a broiler chicken. In the embodiment where the animal is a porcine species, the porcine species can be selected from the group consisting of a grow finish pig, a nursery pig, a sow, and a breeding stock pig. In the embodiment where the animal is a companion animal, the companion animal can be a dog or a cat or any other companion animal. In another embodiment, the methods and compositions described herein are used to treat humans.

In one embodiment, a method of feeding an animal is provided. The method comprises the step of administering to the animal a feed composition or drinking water comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof, wherein the Bacillus strain improves the health of the animal.

In another embodiment, a method of feeding an animal is provided. The method comprises the step of administering to the animal a feed composition or drinking water comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.

In yet another embodiment, a method of improving the health of a human is provided. The method comprises the step of administering to the human a probiotic composition comprising an effective amount of an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.

In various embodiments, the compositions for use in the methods described herein can be a commercial package, a feed additive for an animal feed composition or a human food, an additive for the drinking water of an animal or a human, or an animal feed composition (e.g., a complete feed) or a human food composition, each comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.

The following clauses, and combinations thereof, provide various additional illustrative aspects of the invention described herein. The various embodiments described in any other section of this patent application, including the section titled “DETAILED DESCRIPTION OF ILLUSTRATIVE EMBODIMENTS” and the EXAMPLES are applicable to any of the following embodiments of the invention described in the numbered clauses below.
1. A method of feeding an animal, the method comprising the step of administering to the animal a feed composition or drinking water comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof, wherein the Bacillus strain improves the health of the animal.
2. The method of clause 1 wherein the animal is selected from the group consisting of a poultry species, a porcine species, a bovine species, an ovine species, an equine species, and a companion animal.
3. The method of clause 2 wherein the poultry species is a broiler chicken.
4. The method of any one of clauses 1 to 2 wherein the animal is a companion animal and the companion animal is a dog or a cat.
5. The method of clause 2 wherein the porcine species is selected from the group consisting of a grow finish pig, a nursery pig, a sow, and a breeding stock pig.
6. The method of any one of clauses 1 to 5 wherein the Bacillus strain produces an enzyme selected from the group consisting of an α-galactosidase, a protease, a lipase, an amylase, a xylanase, a cellulase, a methionine reductase, a methionine synthase, a uricase, a prolyl endopeptidase, and combinations thereof.
7. The method of any one of clauses 1 to 6 wherein at least one of the Bacillus strains has antimicrobial activity.
8. The method of clause 7 wherein the antimicrobial activity is against a microbe selected from the group consisting of E. coli, Salmonella, Staphylococcus, Enterococcus, Clostridia, Campylobacter, Candida, Mucor, Penicillium, Aspergillus, and combinations thereof.
9. The method of any one of clauses 1 to 8 further comprising the step of administering to the animal another bacterial strain selected from the group consisting of another different Bacillus strain, a lactic acid bacterial strain, and combinations thereof.
10. The method of any one of clauses 1 to 9 wherein the strain administered is Bacillus strain BC1 (NRRL No. B-67744), or a strain having all of the identifying characteristics of Bacillus strain BC1 (NRRL No. B-67744), or a combination thereof.
11. The method of any one of clauses 1 to 9 wherein the strain administered is Bacillus strain (NRRL No. B-68053), or a strain having all of the identifying characteristics of Bacillus strain (NRRL No. B-68053), or a combination thereof.
12. The method of any one of clauses 1 to 9 wherein the strain administered is Bacillus strain (NRRL No. B-68054), or a strain having all of the identifying characteristics of Bacillus strain (NRRL No. B-68054), or a combination thereof.
13. The method of any one of clauses 1 to 10 wherein the strain administered is Bacillus strain BC1 (NRRL No. B-67744).
14. The method of any one of clauses 1 to 9 wherein the strain administered is Bacillus strain (NRRL No. B-68053).
15. The method of any one of clauses 1 to 9 wherein the strain administered is Bacillus strain (NRRL No. B-68054).
16. The method of any one of clauses 1 to 15 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in a single composition.
17. The method of any one of clauses 1 to 15 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in separate compositions.
18. The method of any one of clauses 1 to 17 wherein the Bacillus strain is administered in the feed composition at a dose of about 1.0×10³ CFU/gram of the feed composition to about 5.0×10¹² CFU/gram of the feed composition.
19. The method of any one of clauses 1 to 17 wherein the Bacillus strain is administered in the feed composition at a dose of about 1.0×10³ CFU/gram of the feed composition to about 1.0×10⁷ CFU/gram of the feed composition.
20. The method of any one of clauses 1 to 17 wherein the Bacillus strain is administered in the feed composition at a dose greater than about 7.0×10⁴ CFU/gram of the feed composition.
21. The method of any one of clauses 1 to 20 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™ chloramphenicol, clindamycin, ciprofloxacin, gentamicin, kanamycin, linezolid, streptomycin, tetracycline, tigecycline, and vancomycin.
22. The method of any one of clauses 1 to 21 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.
23. The method of any one of clauses 1 to 22 wherein the animal is a sow and the Bacillus strain is administered during lactation.
24. The method of any one of clauses 1 to 22 wherein the animal is a sow and the Bacillus strain is administered during gestation.
25. The method of any one of clauses 1 to 22 wherein the feed composition is administered daily to the animal.
26. The method of any one of clauses 1 to 3 or 5 to 25 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.
27. A commercial package comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
28. A feed additive for an animal feed or a human food comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
29. An additive for the drinking water of an animal or a human comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
30. An animal feed composition or a human food composition comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
31. The commercial package, the feed additive for the animal feed or human food, the animal feed composition or the human food composition, or the additive for the drinking water of the animal or the human of any one of clauses 27 to 30 wherein the Bacillus strain improves the health of the animal or the human.
32. The commercial package, the feed additive for the animal feed or human food, the animal feed composition or the human food composition, or the additive for the drinking water of the animal or the human of any one of clauses 27 to 31, wherein the Bacillus strain inhibits a pathogen selected from the group consisting of E. coli, Salmonella, Staphylococcus, Enterococcus, Campylobacter, Clostridium, Candida, Mucor, Penicillium, and Aspergillus.
33. The feed additive for the animal feed or the human food or the additive for the drinking water of the animal or the human of clause 28 or 29 in the form of a concentrate.
34. The feed additive for the animal feed or the human food or the additive for the drinking water of the animal or the human of clause 28 or 29 in the form of a superconcentrate.
35. The feed additive for the animal feed or the human food, the additive for the drinking water of the animal or the human, or the animal feed composition or the human food composition of any one of clauses 28 to 34 in dry form.
36. The feed additive for the animal feed or the human food, the additive for the drinking water of the animal or the human, or the animal feed composition or the human food composition of any one of clauses 28 to 35 in pelleted form.
37. The feed additive for the animal feed or the human food, the additive for the drinking water of the animal or the human, or the animal feed composition or the human food composition of any one of clauses 28 to 35 in powder form.
38. The commercial package, feed additive for the animal feed or the human food, the additive for the drinking water of the animal or the human, or the animal feed composition or the human food composition of any one of clauses 27 to 34 wherein the strains are in a form selected from the group consisting of a powder, a liquid, a gel, a freeze-dried form, a top-dressing, and a pellet form.
39. The commercial package, feed additive for the animal feed or the human food, the additive for the drinking water of the animal or the human, or the animal feed composition or the human food composition of any one of clauses 27 to 38 further comprising a carrier for the Bacillus strains.
40. The commercial package, feed additive for the animal feed or the human food, the additive for the drinking water of the animal or the human, or the animal feed composition or the human food composition of clause 39 wherein the carrier is selected from the group consisting of a bran, rice hulls, a salt, mineral oil, a dextrin, whey, sugar, limestone, dried starch, sodium silico aluminate, vegetable oil, and combinations thereof.
41. The commercial package, feed additive for the animal feed or the human food, the additive for the drinking water of the animal or the human, or the animal feed composition or the human food composition of any one of clauses 27 to 40 in a bag.
42. The commercial package, feed additive for the animal feed or the human food, the additive for the drinking water of the animal or the human, or the animal feed composition or the human food composition of clause 41 wherein the bag is a plastic bag.
43. The commercial package, feed additive for the animal feed or the human food, the additive for the drinking water of the animal or the human, or the animal feed composition or the human food composition of any one of clauses 27 to 42 further comprising instructions for use of one or more of the Bacillus strains.
44. The commercial package, feed additive for the animal feed or the human food, the additive for the drinking water of the animal or the human, or the animal feed composition or the human food composition of any one of clauses 27 to 43 in a 20-pound bag.
45. The commercial package, feed additive for the animal feed or the human food, the additive for the drinking water of the animal or the human, or the animal feed composition or the human food composition of any one of clauses 27 to 43 in a 50-pound bag.
46. The commercial package, feed additive for the animal feed or the human food, the additive for the drinking water of the animal or the human, or the animal feed composition or the human food composition of any one of clauses 27 to 45 in a container for commercial use.
47. The commercial package, feed additive for the animal feed or the human food, the additive for the drinking water of the animal or the human, or the animal feed composition or the human food composition of clause 46 wherein the container comprises plastic.
48. The commercial package, feed additive for the animal feed or the human food, the additive for the drinking water of the animal or the human, or the animal feed composition or the human food composition of clause 46 wherein the container comprises paper.
49. The commercial package, feed additive for the animal feed or the human food, the additive for the drinking water of the animal or the human, or the animal feed composition or the human food composition of any one of clauses 27 to 48 further comprising a binder.
50. The commercial package, feed additive for the animal feed or the human food, the additive for the drinking water of the animal or the human, or the animal feed composition or the human food composition of clause 49 wherein the binder is selected from the group consisting of clay, yeast cell wall components, aluminum silicate, glucan, and combinations thereof.
51. The commercial package, feed additive for the animal feed or the human food, the additive for the drinking water of the animal or the human, or the animal feed composition or the human food composition of any one of clauses 27 to 50 in the form of a dietary nutrient composition.
52. The commercial package, feed additive for the animal feed or the human food, the additive for the drinking water of the animal or the human, or the animal feed composition or the human food composition of any one of clauses 27 to 51 further comprising an exogenously added nutrient component selected from the group consisting of a vitamin, an antibiotic, an enzyme, a water-soluble or water-insoluble monosaccharide, disaccharide, or polysaccharide, a fat, phosphorous, sodium bicarbonate, limestone, calcium, sodium, sulfur, magnesium, potassium, copper, iron, manganese, zinc, fish oil, raw seed, an antioxidant, and a starch.
53. The commercial package, feed additive for the animal feed or the human food, the additive for the drinking water of the animal or the human, or the animal feed composition or the human food composition of clause 52 wherein the exogenously added component is an enzyme.
54. The commercial package, feed additive for the animal feed or the human food, the additive for the drinking water of the animal or the human, or the animal feed composition or the human food composition of clause 53 wherein the enzyme is selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.
55. A method of feeding an animal, the method comprising the step of administering to the animal a feed composition or drinking water comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
56. The method of clause 55 wherein the animal is selected from the group consisting of a poultry species, a porcine species, a bovine species, an ovine species, an equine species, and a companion animal.
57. The method of clause 56 wherein the poultry species is a broiler chicken.
58. The method of clause 56 wherein the porcine species is selected from the group consisting of a grow finish pig, a nursery pig, a sow, and a breeding stock pig.
59. The method of clause 56 wherein the animal is a companion animal and the companion animal is a dog or a cat.
60. The method of any one of clauses 55 to 59 wherein the Bacillus strain produces an enzyme selected from the group consisting of an α-galactosidase, a protease, a lipase, an amylase, a xylanase, a cellulase, a methionine reductase, a methionine synthase, a uricase, a prolyl endopeptidase, and combinations thereof.
61. The method of any one of clauses 55 to 60 wherein at least one of the Bacillus strains has antimicrobial activity.
62. The method of clause 61 wherein the antimicrobial activity is against a microbe selected from the group consisting of E. coli, Salmonella, Staphylococcus, Enterococcus, Clostridia, Campylobacter, Candida, Mucor, Penicillium, Aspergillus, and combinations thereof.
63. The method of any one of clauses 55 to 62 further comprising the step of administering to the animal another bacterial strain selected from the group consisting of another different Bacillus strain, a lactic acid bacterial strain, and combinations thereof.
64. The method of any one of clauses 55 to 63 wherein the strain administered is Bacillus strain BC1 (NRRL No. B-67744), or a strain having all of the identifying characteristics of Bacillus strain BC1 (NRRL No. B-67744), or a combination thereof.
65. The method of any one of clauses 55 to 63 wherein the strain administered is Bacillus strain (NRRL No. B-68053), or a strain having all of the identifying characteristics of Bacillus strain (NRRL No. B-68053), or a combination thereof.
66. The method of any one of clauses 55 to 63 wherein the strain administered is Bacillus strain (NRRL No. B-68054), or a strain having all of the identifying characteristics of Bacillus strain (NRRL No. B-68054), or a combination thereof.
67. The method of any one of clauses 55 to 63 wherein the strain administered is Bacillus strain BC1 (NRRL No. B-67744).
68. The method of any one of clauses 55 to 63 wherein the strain administered is Bacillus strain (NRRL No. B-68053).
69. The method of any one of clauses 55 to 63 wherein the strain administered is Bacillus strain (NRRL No. B-68054).
70. The method of any one of clauses 55 to 63 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in a single composition.
71. The method of any one of clauses 55 to 63 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in separate compositions.
72. The method of any one of clauses 55 to 71 wherein the Bacillus strain is administered in the feed composition at a dose of about 1.0×10³ CFU/gram of the feed composition to about 5.0×10¹² CFU/gram of the feed composition.
73. The method of any one of clauses 55 to 71 wherein the Bacillus strain is administered in the feed composition at a dose of about 1.0×10³ CFU/gram of the feed composition to about 1.0×10⁷ CFU/gram of the feed composition.
74. The method of any one of clauses 55 to 73 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™ chloramphenicol, clindamycin, ciprofloxacin, gentamicin, kanamycin, linezolid, streptomycin, tetracycline, tigecycline, and vancomycin.
75. The method of any one of clauses 55 to 74 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.
76. The method of any one of clauses 55 to 58 or 60 to 75 wherein the animal is a sow and the Bacillus strain is administered during lactation.
77. The method of any one of clauses 55 to 58 or 60 to 75 wherein the animal is a sow and the Bacillus strain is administered during gestation.
78. The method of any one of clauses 55 to 75 wherein the feed composition is administered daily to the animal.
79. The method of any one of clauses 55 to 58 or 60 to 75 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.
80. A method of improving the health of a human, the method comprising the step of administering to the human a probiotic composition comprising an effective amount of an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
81. The method of clause 80 wherein the improvement in health is an improvement in gastrointestinal health.
82. The method of any one of clauses 80 to 81 wherein the Bacillus strain produces an enzyme selected from the group consisting of an α-galactosidase, a protease, a lipase, an amylase, a xylanase, a cellulase, a methionine reductase, a methionine synthase, a uricase, a prolyl endopeptidase, and combinations thereof.
83. The method of any one of clauses 80 to 82 wherein at least one of the Bacillus strains has antimicrobial activity.
84. The method of clause 83 wherein the antimicrobial activity is against a microbe selected from the group consisting of E. coli, Salmonella, Staphylococcus, Enterococcus, Clostridia, Campylobacter, Candida, Mucor, Penicillium, Aspergillus, and combinations thereof.
85. The method of any one of clauses 80 to 84 further comprising the step of administering to the animal another bacterial strain selected from the group consisting of another different Bacillus strain, a lactic acid bacterial strain, and combinations thereof.
86. The method of any one of clauses 80 to 85 wherein the strain administered is Bacillus strain BC1 (NRRL No. B-67744), or a strain having all of the identifying characteristics of Bacillus strain BC1 (NRRL No. B-67744), or a combination thereof.
87. The method of any one of clauses 80 to 85 wherein the strain administered is Bacillus strain (NRRL No. B-68053), or a strain having all of the identifying characteristics of Bacillus strain (NRRL No. B-68053), or a combination thereof.
88. The method of any one of clauses 80 to 85 wherein the strain administered is Bacillus strain (NRRL No. B-68054), or a strain having all of the identifying characteristics of Bacillus strain (NRRL No. B-68054), or a combination thereof.
89. The method of any one of clauses 80 to 85 wherein the strain administered is Bacillus strain BC1 (NRRL No. B-67744).
90. The method of any one of clauses 80 to 85 wherein the strain administered is Bacillus strain (NRRL No. B-68053).
91. The method of any one of clauses 80 to 85 wherein the strain administered is Bacillus strain (NRRL No. B-68054).
92. The method of any one of clauses 80 to 85 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in a single composition.
93. The method of any one of clauses 80 to 85 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in separate compositions.
94. The method of any one of clauses 80 to 93 wherein the Bacillus strain is administered in the probiotic composition at a dose of about 1.0×10³ CFU/gram of the probiotic composition to about 5.0×10¹² CFU/gram of the probiotic composition.
95. The method of any one of clauses 80 to 93 wherein the Bacillus strain is administered in the probiotic composition at a dose of about 1.0×10³ CFU/gram of the probiotic composition to about 1.0×10⁷ CFU/gram of the probiotic composition.
96. The method of any one of clauses 80 to 95 further comprising the step of administering an antibiotic to the human wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™ chloramphenicol, clindamycin, ciprofloxacin, gentamicin, kanamycin, linezolid, streptomycin, tetracycline, tigecycline, and vancomycin.
97. The method of any one of clauses 80 to 96 further comprising the step of administering to the human an enzyme selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.
98. The method of any one of clauses 1 to 26 or 55 to 97 wherein the Bacillus strain does not cause hemolysis.
99. The method of any one of clauses 1 to 26 or 55 to 98 wherein the Bacillus strain does not cause cytotoxicity.
100. The method of any one of clauses 1 to 26 or 55 to 99 wherein the Bacillus strain does not produce a toxin selected from the group consisting of hemolysin BL subunit A (hb1A), hemolysin BL subunit C (hb1C), hemolysin BL subunit D (hb1D), nonhemolytic enterotoxin subunit A (nheA), nonhemolytic enterotoxin subunit B (nheB), nonhemolytic enterotoxin subunit C (nheC), emetic toxin, enterotoxin FM (entFM), enterotoxin T (bceT), and cytotoxin K (cytK).
101. The method of any one of clauses 1 to 26 or 55 to 100 wherein the Bacillus strain produces an anti-inflammatory biomarker selected from the group consisting of SOCS1, TOLLIP, IL-10, and CXCL12.
102. The method of any one of clauses 1 to 26 or 55 to 101 wherein the Bacillus strain has antioxidant activity.
103. The method of any one of clauses 1 to 26 or 55 to 102 wherein the Bacillus strain has quorum quenching activity.
104. The method of any one of clauses 1 to 26 or 55 to 103 wherein the composition or the additive is a dietary nutrient composition.

### BRIEF DESCRIPTION OF THE DRAWINGS

FIGS. 1A-1C show gel electrophoresis results from PCR assays for Bacillus strain BC1 (NRRL No. B-67744). Lanes are identical for each image with Lane 1 for NTC, Lane 2 Bc control, Lane 3 Bt control, and Lane 4 NRRL 67744. FIG. 1A) Master mix 1 containing primer sequences for hb1A, hb1C, hb1D, and emetic strain-specific sequence. FIG. 1B) Master mix 2 containing primer sequences for nheA, nheB, and nheC. FIG. 1C) Master mix 3 containing primer sequences for entFM, bceT, and cytK.

FIG. 2 shows a photograph of a gel displaying RAPD PCR profiles (Primers 1-6) for Bacillus strain BC1 (NRRL No. B-67744). The leftmost and rightmost lanes include markers and each set of two consecutive lanes between the markers from left to right correspond to Primers 1-6.

FIGS. 3A-3C show relative quantification of expression of inflammatory cytokines and pathogen receptors IL-6 (FIG. 3A), TLR2 (FIG. 3B) and TNF (FIG. 3C) in the IEC-6 cell line challenged with 10 ng LPS and treated with Bacillus coagulans NRRL 67744 at 10³, 10⁴, and 10⁵ CFU/ml. Different letters indicate significant differences (p<0.05).

FIGS. 4A-4C show gel electrophoresis results from the PCR assays. Lanes are identical for each image with Lane 1 for NTC, Lane 2 Bc control, Lane 3 Bt control, and Lane 4 NRRL 68053. FIG. 4A) Master mix 1 containing primer sequences for hb1A, hb1C, hb1D, and emetic strain-specific sequence. FIG. 4B) Master mix 2 containing primer sequences for nheA, nheB, and nheC. FIG. 4C) Master mix 3 containing primer sequences for entFM, bceT, and cytK.

FIG. 5 shows RAPD-PCR gel electrophoresis results for NRRL No. B-68053. Lanes 1 and 8 are molecular weight ladders with 200 kb increments. For each of the six primers in the PCR reaction, the strain DNA was run in duplicates. Lanes 2 and 3 are primer 1. Lanes 4 and 5 are primer 2. Lanes 6 and 7 are primer 3. Lanes 9 and 10 are primer 4. Lanes 11 and 12 are primer 5. Lanes 13 and 14 are primer 6.

FIG. 6 shows RAPD-PCR gel electrophoresis results for NRRL No. B-68054. Lanes 1 and 8 are molecular weight ladders with 200 kb increments. For each of the six primers in the PCR reaction, the strain DNA was run in duplicates. Lanes 2 and 3 are primer 1. Lanes 4 and 5 are primer 2. Lanes 6 and 7 are primer 3. Lanes 9 and 10 are primer 4. Lanes 11 and 12 are primer 5. Lanes 13 and 14 are primer 6.

FIGS. 7A-7C show gel electrophoresis results from the PCR assays. Lanes are identical for each image with Lane 1 for NTC, Lane 2 Bc control, Lane 3 Bt control, and Lane 4 NRRL 68054. FIG. 7A) Master mix 1 containing primer sequences for hb1A, hb1C, hb1D, and emetic strain-specific sequence. FIG. 7B) Master mix 2 containing primer sequences for nheA, nheB, and nheC. FIG. 7C) Master mix 3 containing primer sequences for entFM, bceT, and cytK.

### DETAILED DESCRIPTION OF ILLUSTRATIVE EMBODIMENTS

Methods and compositions are provided for improving health in animals and humans. In various embodiments, the animal can be selected from the group consisting of a poultry species, a porcine species, a bovine species, an ovine species, an equine species, and a companion animal. In the embodiment where the animal is a poultry species, the poultry species can be a broiler chicken. In the embodiment where the animal is a porcine species, the porcine species can be selected from the group consisting of a grow finish pig, a nursery pig, a sow, and a breeding stock pig. In the embodiment where the animal is a companion animal, the companion animal can be a dog or a cat or any other companion animal. In another embodiment, the methods and compositions described herein are used to treat humans.

In one embodiment, a method of feeding an animal is provided. The method comprises the step of administering to the animal a feed composition or drinking water comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof, wherein the Bacillus strain improves the health of the animal.

In another embodiment, a method of feeding an animal is provided. The method comprises the step of administering to the animal a feed composition or drinking water comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.

In yet another embodiment, a method of improving the health of a human is provided. The method comprises the step of administering to the human a probiotic composition comprising an effective amount of an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.

In various embodiments, the compositions for use in the methods described herein can be a commercial package, a feed additive for an animal feed composition or a human food, an additive for the drinking water of an animal or a human, or an animal feed composition (e.g., a complete feed) or a human food composition, each comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.

The following clauses, and combinations thereof, provide various additional illustrative aspects of the invention described herein. The various embodiments described in this section titled “DETAILED DESCRIPTION OF ILLUSTRATIVE EMBODIMENTS” are applicable to any of the following embodiments of the invention described in the numbered clauses below.
1. A method of feeding an animal, the method comprising the step of administering to the animal a feed composition or drinking water comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof, wherein the Bacillus strain improves the health of the animal.
2. The method of clause 1 wherein the animal is selected from the group consisting of a poultry species, a porcine species, a bovine species, an ovine species, an equine species, and a companion animal.
3. The method of clause 2 wherein the poultry species is a broiler chicken.
4. The method of any one of clauses 1 to 2 wherein the animal is a companion animal and the companion animal is a dog or a cat.
5. The method of clause 2 wherein the porcine species is selected from the group consisting of a grow finish pig, a nursery pig, a sow, and a breeding stock pig.
6. The method of any one of clauses 1 to 5 wherein the Bacillus strain produces an enzyme selected from the group consisting of an α-galactosidase, a protease, a lipase, an amylase, a xylanase, a cellulase, a methionine reductase, a methionine synthase, a uricase, a prolyl endopeptidase, and combinations thereof.
7. The method of any one of clauses 1 to 6 wherein at least one of the Bacillus strains has antimicrobial activity.
8. The method of clause 7 wherein the antimicrobial activity is against a microbe selected from the group consisting of E. coli, Salmonella, Staphylococcus, Enterococcus, Clostridia, Campylobacter, Candida, Mucor, Penicillium, Aspergillus, and combinations thereof.
9. The method of any one of clauses 1 to 8 further comprising the step of administering to the animal another bacterial strain selected from the group consisting of another different Bacillus strain, a lactic acid bacterial strain, and combinations thereof.
10. The method of any one of clauses 1 to 9 wherein the strain administered is Bacillus strain BC1 (NRRL No. B-67744), or a strain having all of the identifying characteristics of Bacillus strain BC1 (NRRL No. B-67744), or a combination thereof.
11. The method of any one of clauses 1 to 9 wherein the strain administered is Bacillus strain (NRRL No. B-68053), or a strain having all of the identifying characteristics of Bacillus strain (NRRL No. B-68053), or a combination thereof.
12. The method of any one of clauses 1 to 9 wherein the strain administered is Bacillus strain (NRRL No. B-68054), or a strain having all of the identifying characteristics of Bacillus strain (NRRL No. B-68054), or a combination thereof.

13. The method of any one of clauses 1 to 10 wherein the strain administered is Bacillus strain BC1 (NRRL No. B-67744).
14. The method of any one of clauses 1 to 9 wherein the strain administered is Bacillus strain (NRRL No. B-68053).
15. The method of any one of clauses 1 to 9 wherein the strain administered is Bacillus strain (NRRL No. B-68054).
16. The method of any one of clauses 1 to 15 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in a single composition.
17. The method of any one of clauses 1 to 15 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in separate compositions.
18. The method of any one of clauses 1 to 17 wherein the Bacillus strain is administered in the feed composition at a dose of about 1.0×10³ CFU/gram of the feed composition to about 5.0×10¹² CFU/gram of the feed composition.
19. The method of any one of clauses 1 to 17 wherein the Bacillus strain is administered in the feed composition at a dose of about 1.0×10³ CFU/gram of the feed composition to about 1.0×10⁷ CFU/gram of the feed composition.
20. The method of any one of clauses 1 to 17 wherein the Bacillus strain is administered in the feed composition at a dose greater than about 7.0×10⁴ CFU/gram of the feed composition.
21. The method of any one of clauses 1 to 20 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™ chloramphenicol, clindamycin, ciprofloxacin, gentamicin, kanamycin, linezolid, streptomycin, tetracycline, tigecycline, and vancomycin.
22. The method of any one of clauses 1 to 21 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.
23. The method of any one of clauses 1 to 22 wherein the animal is a sow and the Bacillus strain is administered during lactation.
24. The method of any one of clauses 1 to 22 wherein the animal is a sow and the Bacillus strain is administered during gestation.
25. The method of any one of clauses 1 to 22 wherein the feed composition is administered daily to the animal.
26. The method of any one of clauses 1 to 3 or 5 to 25 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.
27. A commercial package comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
28. A feed additive for an animal feed or a human food comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
29. An additive for the drinking water of an animal or a human comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
30. An animal feed composition or a human food composition comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
31. The commercial package, the feed additive for the animal feed or human food, the animal feed composition or the human food composition, or the additive for the drinking water of the animal or the human of any one of clauses 27 to 30 wherein the Bacillus strain improves the health of the animal or the human.
32. The commercial package, the feed additive for the animal feed or human food, the animal feed composition or the human food composition, or the additive for the drinking water of the animal or the human of any one of clauses 27 to 31, wherein the Bacillus strain inhibits a pathogen selected from the group consisting of E. coli, Salmonella, Staphylococcus, Enterococcus, Campylobacter, Clostridium, Candida, Mucor, Penicillium, and Aspergillus.
33. The feed additive for the animal feed or the human food or the additive for the drinking water of the animal or the human of clause 28 or 29 in the form of a concentrate.
34. The feed additive for the animal feed or the human food or the additive for the drinking water of the animal or the human of clause 28 or 29 in the form of a superconcentrate.
35. The feed additive for the animal feed or the human food, the additive for the drinking water of the animal or the human, or the animal feed composition or the human food composition of any one of clauses 28 to 34 in dry form.
36. The feed additive for the animal feed or the human food, the additive for the drinking water of the animal or the human, or the animal feed composition or the human food composition of any one of clauses 28 to 35 in pelleted form.
37. The feed additive for the animal feed or the human food, the additive for the drinking water of the animal or the human, or the animal feed composition or the human food composition any one of clauses 28 to 35 in powder form.
38. The commercial package, feed additive for the animal feed or the human food, the additive for the drinking water of the animal or the human, or the animal feed composition or the human food composition of any one of clauses 27 to 34 wherein the strains are in a form selected from the group consisting of a powder, a liquid, a gel, a freeze-dried form, a top-dressing, and a pellet form.
39. The commercial package, feed additive for the animal feed or the human food, the additive for the drinking water of the animal or the human, or the animal feed composition or the human food composition of any one of clauses 27 to 38 further comprising a carrier for the Bacillus strains.
40. The commercial package, feed additive for the animal feed or the human food, the additive for the drinking water of the animal or the human, or the animal feed composition or the human food composition of clause 39 wherein the carrier is selected from the group consisting of a bran, rice hulls, a salt, mineral oil, a dextrin, whey, sugar, limestone, dried starch, sodium silico aluminate, vegetable oil, and combinations thereof.
41. The commercial package, feed additive for the animal feed or the human food, the additive for the drinking water of the animal or the human, or the animal feed composition or the human food composition of any one of clauses 27 to 40 in a bag.
42. The commercial package, feed additive for the animal feed or the human food, the additive for the drinking water of the animal or the human, or the animal feed composition or the human food composition of clause 41 wherein the bag is a plastic bag.
43. The commercial package, feed additive for the animal feed or the human food, the additive for the drinking water of the animal or the human, or the animal feed composition or the human food composition of any one of clauses 27 to 42 further comprising instructions for use of one or more of the Bacillus strains.
44. The commercial package, feed additive for the animal feed or the human food, the additive for the drinking water of the animal or the human, or the animal feed composition or the human food composition of any one of clauses 27 to 43 in a 20-pound bag.
45. The commercial package, feed additive for the animal feed or the human food, the additive for the drinking water of the animal or the human, or the animal feed composition or the human food composition of any one of clauses 27 to 43 in a 50-pound bag.
46. The commercial package, feed additive for the animal feed or the human food, the additive for the drinking water of the animal or the human, or the animal feed composition or the human food composition of any one of clauses 27 to 45 in a container for commercial use.
47. The commercial package, feed additive for the animal feed or the human food, the additive for the drinking water of the animal or the human, or the animal feed composition or the human food composition of clause 46 wherein the container comprises plastic.
48. The commercial package, feed additive for the animal feed or the human food, the additive for the drinking water of the animal or the human, or the animal feed composition or the human food composition of clause 46 wherein the container comprises paper.
49. The commercial package, feed additive for the animal feed or the human food, the additive for the drinking water of the animal or the human, or the animal feed composition or the human food composition of any one of clauses 27 to 48 further comprising a binder.
50. The commercial package, feed additive for the animal feed or the human food, the additive for the drinking water of the animal or the human, or the animal feed composition or the human food composition of clause 49 wherein the binder is selected from the group consisting of clay, yeast cell wall components, aluminum silicate, glucan, and combinations thereof.
51. The commercial package, feed additive for the animal feed or the human food, the additive for the drinking water of the animal or the human, or the animal feed composition or the human food composition of any one of clauses 27 to 50 in the form of a dietary nutrient composition.
52. The commercial package, feed additive for the animal feed or the human food, the additive for the drinking water of the animal or the human, or the animal feed composition or the human food composition of any one of clauses 27 to 51 further comprising an exogenously added nutrient component selected from the group consisting of a vitamin, an antibiotic, an enzyme, a water-soluble or water-insoluble monosaccharide, disaccharide, or polysaccharide, a fat, phosphorous, sodium bicarbonate, limestone, calcium, sodium, sulfur, magnesium, potassium, copper, iron, manganese, zinc, fish oil, raw seed, an antioxidant, and a starch.
53. The commercial package, feed additive for the animal feed or the human food, the additive for the drinking water of the animal or the human, or the animal feed composition or the human food composition of clause 52 wherein the exogenously added component is an enzyme.
54. The commercial package, feed additive for the animal feed or the human food, the additive for the drinking water of the animal or the human, or the animal feed composition or the human food composition of clause 53 wherein the enzyme is selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.
55. A method of feeding an animal, the method comprising the step of administering to the animal a feed composition or drinking water comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
56. The method of clause 55 wherein the animal is selected from the group consisting of a poultry species, a porcine species, a bovine species, an ovine species, an equine species, and a companion animal.
57. The method of clause 56 wherein the poultry species is a broiler chicken.
58. The method of clause 56 wherein the porcine species is selected from the group consisting of a grow finish pig, a nursery pig, a sow, and a breeding stock pig.
59. The method of clause 56 wherein the animal is a companion animal and the companion animal is a dog or a cat.
60. The method of any one of clauses 55 to 59 wherein the Bacillus strain produces an enzyme selected from the group consisting of an α-galactosidase, a protease, a lipase, an amylase, a xylanase, a cellulase, a methionine reductase, a methionine synthase, a uricase, a prolyl endopeptidase, and combinations thereof.
61. The method of any one of clauses 55 to 60 wherein at least one of the Bacillus strains has antimicrobial activity.
62. The method of clause 61 wherein the antimicrobial activity is against a microbe selected from the group consisting of E. coli, Salmonella, Staphylococcus, Enterococcus, Clostridia, Campylobacter, Candida, Mucor, Penicillium, Aspergillus, and combinations thereof.
63. The method of any one of clauses 55 to 62 further comprising the step of administering to the animal another bacterial strain selected from the group consisting of another different Bacillus strain, a lactic acid bacterial strain, and combinations thereof.
64. The method of any one of clauses 55 to 63 wherein the strain administered is Bacillus strain BC1 (NRRL No. B-67744), or a strain having all of the identifying characteristics of Bacillus strain BC1 (NRRL No. B-67744), or a combination thereof.
65. The method of any one of clauses 55 to 63 wherein the strain administered is Bacillus strain (NRRL No. B-68053), or a strain having all of the identifying characteristics of Bacillus strain (NRRL No. B-68053), or a combination thereof.
66. The method of any one of clauses 55 to 63 wherein the strain administered is Bacillus strain (NRRL No. B-68054), or a strain having all of the identifying characteristics of Bacillus strain (NRRL No. B-68054), or a combination thereof.
67. The method of any one of clauses 55 to 63 wherein the strain administered is Bacillus strain BC1 (NRRL No. B-67744).
68. The method of any one of clauses 55 to 63 wherein the strain administered is Bacillus strain (NRRL No. B-68053).
69. The method of any one of clauses 55 to 63 wherein the strain administered is Bacillus strain (NRRL No. B-68054).
70. The method of any one of clauses 55 to 63 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in a single composition.
71. The method of any one of clauses 55 to 63 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in separate compositions.
72. The method of any one of clauses 55 to 71 wherein the Bacillus strain is administered in the feed composition at a dose of about 1.0×10³ CFU/gram of the feed composition to about 5.0×10¹² CFU/gram of the feed composition.
73. The method of any one of clauses 55 to 71 wherein the Bacillus strain is administered in the feed composition at a dose of about 1.0×10³ CFU/gram of the feed composition to about 1.0×10⁷ CFU/gram of the feed composition.
74. The method of any one of clauses 55 to 73 further comprising the step of administering an antibiotic to the animal wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™ chloramphenicol, clindamycin, ciprofloxacin, gentamicin, kanamycin, linezolid, streptomycin, tetracycline, tigecycline, and vancomycin.
75. The method of any one of clauses 55 to 74 further comprising the step of administering to the animal an enzyme selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.
76. The method of any one of clauses 55 to 58 or 60 to 75 wherein the animal is a sow and the Bacillus strain is administered during lactation.
77. The method of any one of clauses 55 to 58 or 60 to 75 wherein the animal is a sow and the Bacillus strain is administered during gestation.
78. The method of any one of clauses 55 to 75 wherein the feed composition is administered daily to the animal.
79. The method of any one of clauses 55 to 58 or 60 to 75 wherein the animal is selected from the group consisting of a chicken, a pig, a horse, a pony, a cow, a turkey, a goat, a sheep, a quail, a pheasant, an ostrich, a duck, a fish, a crustacean, and combinations thereof.
80. A method of improving the health of a human, the method comprising the step of administering to the human a probiotic composition comprising an effective amount of an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.
81. The method of clause 80 wherein the improvement in health is an improvement in gastrointestinal health.
82. The method of any one of clauses 80 to 81 wherein the Bacillus strain produces an enzyme selected from the group consisting of an α-galactosidase, a protease, a lipase, an amylase, a xylanase, a cellulase, a methionine reductase, a methionine synthase, a uricase, a prolyl endopeptidase, and combinations thereof.
83. The method of any one of clauses 80 to 82 wherein at least one of the Bacillus strains has antimicrobial activity.
84. The method of clause 83 wherein the antimicrobial activity is against a microbe selected from the group consisting of E. coli, Salmonella, Staphylococcus, Enterococcus, Clostridia, Campylobacter, Candida, Mucor, Penicillium, Aspergillus, and combinations thereof.
85. The method of any one of clauses 80 to 84 further comprising the step of administering to the animal another bacterial strain selected from the group consisting of another different Bacillus strain, a lactic acid bacterial strain, and combinations thereof.
86. The method of any one of clauses 80 to 85 wherein the strain administered is Bacillus strain BC1 (NRRL No. B-67744), or a strain having all of the identifying characteristics of Bacillus strain BC1 (NRRL No. B-67744), or a combination thereof.
87. The method of any one of clauses 80 to 85 wherein the strain administered is Bacillus strain (NRRL No. B-68053), or a strain having all of the identifying characteristics of Bacillus strain (NRRL No. B-68053), or a combination thereof.
88. The method of any one of clauses 80 to 85 wherein the strain administered is Bacillus strain (NRRL No. B-68054), or a strain having all of the identifying characteristics of Bacillus strain (NRRL No. B-68054), or a combination thereof.
89. The method of any one of clauses 80 to 85 wherein the strain administered is Bacillus strain BC1 (NRRL No. B-67744).
90. The method of any one of clauses 80 to 85 wherein the strain administered is Bacillus strain (NRRL No. B-68053).
91. The method of any one of clauses 80 to 85 wherein the strain administered is Bacillus strain (NRRL No. B-68054).
92. The method of any one of clauses 80 to 85 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in a single composition.
93. The method of any one of clauses 80 to 85 wherein at least two of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), or combinations thereof, are administered in combination in separate compositions.
94. The method of any one of clauses 80 to 93 wherein the Bacillus strain is administered in the probiotic composition at a dose of about 1.0×10³ CFU/gram of the probiotic composition to about 5.0×10¹² CFU/gram of the probiotic composition.
95. The method of any one of clauses 80 to 93 wherein the Bacillus strain is administered in the probiotic composition at a dose of about 1.0×10³ CFU/gram of the probiotic composition to about 1.0×10⁷ CFU/gram of the probiotic composition.
96. The method of any one of clauses 80 to 95 further comprising the step of administering an antibiotic to the human wherein the antibiotic is selected from the group consisting of Denagard™, BMD™, Carbadox™, Stafac™, erythromycin, levofloxacin, trimethoprim/sulfamethoxazole, trimethoprim, daptomycin, rifampicin, Tylan™, Pulmotil™ chloramphenicol, clindamycin, ciprofloxacin, gentamicin, kanamycin, linezolid, streptomycin, tetracycline, tigecycline, and vancomycin.
97. The method of any one of clauses 80 to 96 further comprising the step of administering to the human an enzyme selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.
98. The method of any one of clauses 1 to 26 or 55 to 97 wherein the Bacillus strain does not cause hemolysis.
99. The method of any one of clauses 1 to 26 or 55 to 98 wherein the Bacillus strain does not cause cytotoxicity.
100. The method of any one of clauses 1 to 26 or 55 to 99 wherein the Bacillus strain does not produce a toxin selected from the group consisting of hemolysin BL subunit A (hb1A), hemolysin BL subunit C (hb1C), hemolysin BL subunit D (hb1D), nonhemolytic enterotoxin subunit A (nheA), nonhemolytic enterotoxin subunit B (nheB), nonhemolytic enterotoxin subunit C (nheC), emetic toxin, enterotoxin FM (entFM), enterotoxin T (bceT), and cytotoxin K (cytK).
101. The method of any one of clauses 1 to 26 or 55 to 100 wherein the Bacillus strain produces an anti-inflammatory biomarker selected from the group consisting of SOCS1, TOLLIP, IL-10, and CXCL12.
102. The method of any one of clauses 1 to 26 or 55 to 101 wherein the Bacillus strain has antioxidant activity.
103. The method of any one of clauses 1 to 26 or 55 to 102 wherein the Bacillus strain has quorum quenching activity.
104. The method of any one of clauses 1 to 26 or 55 to 103 wherein the composition or the additive is a dietary nutrient composition.

In various embodiments, the animal to which a feed additive, a feed composition, or drinking water as described herein is administered can be selected from the group consisting of a poultry species, a porcine species, a bovine species, an ovine species, an equine species, and a companion animal. In the embodiment where the animal is a companion animal, the companion animal can be, for example, a canine species or a feline species. In the embodiment where the animal is a porcine species, the porcine species can be selected from the group consisting of a grow finish pig, a nursery pig, a sow, and a breeding stock pig. In various exemplary embodiments, the animal can be selected from the group consisting of a chicken (e.g., a broiler or a layer), a pig, a horse, a pony, a cow, a turkey, a goat, a sheep, a quail, a pheasant, an ostrich, a duck, a fish (e.g., a tilapia, a catfish, a flounder, or a salmon), a crustacean (e.g., a shrimp or a crab), and combinations thereof.

In another embodiment, the additive for food, the food composition, the consumable liquid, or drinking water comprising the probiotic composition(s) described herein is administered to a human. As used herein “administered”, “administering”, “administer”, “administration”, and the like means administration by another person or that the human consumes the probiotic composition(s) described herein on their own. As used herein in reference to consumption by a human, “a consumable liquid” means any consumable liquid including water, sports nutrition drinks or other nutritional beverages, soft drinks, juices, teas, coffee, milk, lactic acid bacteria drinks, and the like. In various embodiments described herein, the probiotic composition can also be referred to as a “direct-fed microbial composition” and both types of compositions can be dietary nutrient compositions. In one embodiment, the commercial package described herein can contain a probiotic composition, a direct-fed microbial composition, or a dietary nutrient composition comprising the Bacillus strains described herein.

In one embodiment of the invention, an effective amount of the Bacillus strain can be administered to improve the health of the animal or the human. By “effective amount” is meant an amount of the Bacillus strain (e.g., Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof) capable of improving the health of an animal or a human by any mechanism, including those described herein.

In embodiments described herein wherein the compositions of the present invention comprising Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, are administered to an animal or a human, the compositions are preferably administered to animals orally in a feed composition or in drinking water, or orally to a human in food or a consumable liquid or drinking water but any other effective method of administration known to those skilled in the art may be utilized. In one illustrative embodiment, the Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, are provided in the form of an additive for addition to an animal's feed composition or to the drinking water of an animal or to food, to a consumable liquid, or to drinking water for a human.

In one illustrative embodiment, the Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, are provided in the form of a feed additive for addition to a feed composition for an animal. The feed composition may contain Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, in a mixture with an animal feed blend, including any art-recognized animal feed blend or any animal feed blend described herein. As used herein, “feed composition” or “animal feed composition” means a feed composition comprising Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, in a mixture with an animal feed blend, and, optionally any other components that could be used in a feed composition, including other different bacterial strains, such as other Bacillus strains or Lactobacillus strains.

Any animal feed blend, including those known in the art and those described herein, may be used in accordance with the methods and compositions described in this patent application, such as rapeseed meal, cottonseed meal, soybean meal, cornmeal, barley, wheat, silage, and haylage. In various embodiments, the animal feed blend can be supplemented with Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, but other ingredients may optionally be added to the animal feed blend.

In another illustrative aspect, any medicament ingredients known in the art may be added to the animal feed blend or to an additive for the drinking water of the animal, such as antibiotics. In various embodiments, the antibiotic is selected from the group consisting of ampicillin, chloramphenicol, ciprofloxacin, clindamycin, tetracycline, chlortetracycline, Denagard™ (i.e., tiamulin), BMD™ (i.e., bacitracin methylene disalicylate), Carbadox™ (i.e., carbadox), Stafac™ (i.e., virginiamycin), erythromycin, levofloxacin, trimethoprim/sulfamethoxazole, trimethoprim, daptomycin, rifampicin, Tylan™ (i.e., tylosin), Pulmotil™ (i.e., tilmicosin), vancomycin, avilamycin (Kavault™), gentamycin, neomycin, ciprofloxacin, kanamycin, linezolid, streptomycin, tigecycline, and combinations thereof. In another embodiment, the animal feed blend, the feed composition, the feed additive, or the additive for the drinking water of the animal may contain no antibiotics.

In the embodiment where the Bacillus strains are included in a food composition for human consumption either as an additive for food or for a consumable liquid or for drinking water, or by direct consumption, the Bacillus strains may be added to such foods and consumable liquids as yogurt, beverages such as water, sports nutrition drinks or other nutritional beverages, beverage powders, soft drinks, juices, teas, coffee, milk, lactic acid bacteria drinks, cheese, ice cream, desserts and any dessert product such as fruit or cream fillings, icings, or cheese cake filling, bread products, such as sandwich breads, biscuits, cake mixes, rolls, muffins, and any other food or consumable liquid for human consumption that is suitable for addition of the Bacillus strains described herein in the form of a probiotic composition. In another embodiment, the Bacillus strains described herein may be consumed directly by a human in the form of, for example, a tablet, a capsule, a gel, a gelatin capsule, a powder, granules, a liquid, a spray, or any other suitable form for direct consumption by a human. Optionally any other components that could be used in a food composition or a consumable liquid may be added, including other different bacterial strains, such as other Bacillus strains or Lactobacillus strains.

In various illustrative embodiments, optional ingredients of the animal feed blend or the food composition include sugars and complex carbohydrates such as both water-soluble and water-insoluble monosaccharides, disaccharides, and polysaccharides. Other optional ingredients include dried distillers grain solubles, fat (e.g., crude fat), phosphorous, sodium bicarbonate, limestone, salt, phytate, calcium, sodium, sulfur, magnesium, potassium, copper, iron, manganese, zinc, ash, fish oil, an oil derived from fish meal, raw seed (e.g., flaxseed), an antioxidant, and starch. In another embodiment, minerals may be added in the form of a mineral premix.

Optional amino acid ingredients that may be added to the animal feed blend or the food composition are arginine, histidine, isoleucine, leucine, lysine, cysteine, methionine, phenylalanine, threonine, tryptophan, valine, tyrosine ethyl HCl, alanine, aspartic acid, sodium glutamate, glycine, proline, serine, cysteine ethyl HCl, and analogs, and salts thereof. Vitamins that may be optionally added are thiamine HCl, riboflavin, pyridoxine HCl, niacin, niacinamide, inositol, choline chloride, calcium pantothenate, biotin, folic acid, ascorbic acid, and vitamins A, B, K, D, E, and the like. In another embodiment, vitamins may be added in the form of a vitamin premix. In yet another embodiment, protein ingredients may be added to the animal feed blend or the food composition and include protein obtained from meat meal, bone meal, or fish meal, liquid or powdered egg, fish solubles, crude protein, and the like.

In another illustrative embodiment, one or more enzymes may be added to the animal feed blend or the human food or the consumable liquid. In various embodiments, the enzymes that may be added include a galactosidase, a phytase, a protease, a lipase, an amylase, a hemicellulase, an arabinoxylanase, a xylanase, a cellulase, an NSPase, a methionine reductase, a methionine synthase, a uricase, a prolyl endopeptidase, combinations thereof, and any other enzyme that improves the effectiveness of the feed composition, the food, or the consumable liquid for improving the performance or health of the animal or the human. In yet another embodiment, yeast, fungi (e.g., Aspergillus or Trichoderma), or micronutrients may be added to the animal feed or the human food. Any of the ingredients described above that are suitable for addition to an additive for the drinking water of the animal or the human or to a consumable liquid may be added as a component of the additive for the drinking water of the animal or the human or to the consumable liquid as described herein.

In various illustrative embodiments, the Bacillus strain (e.g., Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof), or any other bacterial strains added in addition to Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, can be administered in the animal feed composition or a human food composition at a dose of about 1.0×10³ CFU/gram of the feed composition or the human food composition to about 5.0×10¹² CFU/gram of the feed composition or food composition or at a dose of about 1.0×10³ CFU/gram of the feed composition or the food composition to about 1.0×10⁷ CFU/gram of the feed composition or the food composition. In other embodiments, the Bacillus strain (e.g., Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof) is administered in the feed composition or the food composition at a dose greater than about 1.0×10³ CFU/gram of the feed composition or the food composition, at a dose greater than about 1.1×10³ CFU/gram of the feed composition or the food composition, at a dose greater than about 1.25×10³ CFU/gram of the feed composition or the food composition, at a dose greater than about 1.5×10³ CFU/gram of the feed composition or the food composition, at a dose greater than about 1.75×10³ CFU/gram of the feed composition or the food composition, at a dose greater than about 1.0×10⁴ CFU/gram of the feed composition or the food composition, at a dose greater than about 2.0×10⁴ CFU/gram of the feed composition or the food composition, at a dose greater than about 3.0×10⁴ CFU/gram of the feed composition or the food composition, at a dose greater than about 4.0×10⁴ CFU/gram of the feed composition or the food composition, at a dose greater than about 5.0×10⁴ CFU/gram of the feed composition or the food composition, at a dose greater than about 6.0×10⁴ CFU/gram of the feed composition or the food composition, at a dose greater than about 7.0×10⁴ CFU/gram of the feed composition or the food composition, at a dose greater than about 8.0×10⁴ CFU/gram of the feed composition or the food composition, at a dose greater than about 1.0×10⁵ CFU/gram of the feed composition or the food composition, at a dose greater than about 1.0×10⁶ CFU/gram of the feed composition or the food composition, at a dose greater than about 1.0×10⁷ CFU/gram of the feed composition or the food composition, at a dose greater than about 1.0×10⁸ CFU/gram of the feed composition or the food composition, at a dose greater than about 1.0×10⁹ CFU/gram of the feed composition or the food composition, at a dose greater than about 1.0×10¹⁰ CFU/gram of the feed composition or the food composition, at a dose greater than about 1.0×10¹¹ CFU/gram of the feed composition or the food composition, or at a dose greater than about 1.0×10¹² CFU/gram of the feed composition or the food composition. In yet another embodiment, the Bacillus strain (e.g., Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof) is administered in the feed composition or the food composition at a dose of about 7×10⁴ CFU/gram of the feed composition or the food composition. In another embodiment, any of the dosages described herein can be in CFU/ml of drinking water or a consumable liquid in embodiments where the strains are administered in the drinking water of the animal, or the drinking water or a consumable liquid for a human.

In another embodiment, the dose of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054) can be about 10⁵ CFU/day, about 10⁶ CFU/day, about 10⁷ CFU/day, about 10⁸ CFU/day, about 10⁹ CFU/day, about 10¹⁰ CFU/day, about 10¹¹ CFU/day, or about 10¹² CFU/day, or any suitable amount of the strain in CFU/day.

In various embodiments, the Bacillus strain for use in accordance with the methods and compositions described herein can be selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof. Bacillus strain MDGBC1 (strain BC1) was deposited on Feb. 6, 2019 at the Agricultural Research Service Culture Collection (NRRL), National Center for Agricultural Utilization Research, Agricultural Research Service, USDA, 1815 North University Street, Peoria, Illinois 61604-3999, and was given accession number B-67744. Bacillus strains MDG-HP20 and MDG-HP29 were deposited on Jul. 6, 2021 at the Agricultural Research Service Culture Collection (NRRL), National Center for Agricultural Utilization Research, Agricultural Research Service, USDA, 1815 North University Street, Peoria, Illinois 61604-3999, and were given accession numbers B-68053 and B-68054, respectively. The deposits were made under the provisions of the Budapest Treaty on the International Recognition of the Deposit of Microorganisms for the Purposes of Patent Procedure. The NRRL strain designations are MDGBC1, MDG-HP20, and MDG-HP29, which are equivalent to Bacillus coagulans strain BC1 and Bacillus subtilis strains NRRL No. B-68053 and NRRL No. B-68054, respectively, as referred to in this application.

Any of these strains can be administered alone or in combination in the form of a feed composition (e.g., a complete feed comprising an animal feed blend), or a food composition for a human, or drinking water for an animal or a human, or a consumable liquid, or the strains can be administered directly without any added feed or food ingredients. In one embodiment, multiple strains are administered in combination in a single composition. In another embodiment, multiple strains are administered in combination in separate compositions.

In another embodiment, one or more of the Bacillus strains described in the preceding paragraphs (e.g., Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof) can be administered to the animal or the human along with another different bacterial strain selected from the group consisting of another Bacillus strain, a lactic acid bacterial strain, and combinations thereof. In yet another embodiment, one or more of the Bacillus strains described in the preceding paragraphs (e.g., Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof) can be administered to the animal or the human along with any other different bacterial strain effective to improve the performance or health of the animal or the human.

As used herein “a strain having all of the identifying characteristics of” Bacillus strain BC1 (NRRL No. B-67744) or Bacillus strain (NRRL No. B-68053) or Bacillus strain (NRRL No. B-68054) can be a mutant strain having all of the identifying characteristics of these Bacillus strains (e.g., a DNA fingerprint based on DNA analysis that corresponds to the DNA fingerprint of these strains, enzyme activities that correspond to these strains, antimicrobial activity that corresponds to these strains, antibiotic sensitivity and tolerance profiles that correspond to these strains, or combinations thereof). In alternate embodiments, the mutation can be a natural mutation, or a genetically engineered mutation. In another embodiment, “a strain having all of the identifying characteristics of” Bacillus strain BC1 or Bacillus strain (NRRL B-68053) or Bacillus strain (NRRL B-68054) can be a strain, for example, produced by isolating one or more plasmids from Bacillus strain BC1 or Bacillus strain (NRRL B-68053) or Bacillus strain (NRRL B-68054) and introducing the one or more plasmids into another bacterium, such as another Bacillus strain, as long as the one or more plasmids contain DNA that provides the identifying characteristics of Bacillus strain BC1 or Bacillus strain (NRRL B-68053) or Bacillus strain (NRRL B-68054) (e.g., a DNA fingerprint based on DNA analysis that corresponds to the DNA fingerprint of Bacillus strain BC1 or Bacillus strain (NRRL B-68053) or Bacillus strain (NRRL B-68054)).

The feed composition or drinking water for an animal or the food composition or drinking water or consumable liquid for a human comprising Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, may be administered to the animal or the human for any time period that is effective to improve the health of the animal or the human. For example, in one embodiment the feed or food composition or drinking water or consumable liquid may be provided to the animal or the human daily. In an alternate embodiment, the feed composition or drinking water may be administered to the animal during lactation and/or during gestation. The time periods for administration of the feed or food composition or drinking water or consumable liquid described above are non-limiting examples and it should be appreciated that any time period or administration schedule determined to be effective to improve the health of the animal or the human may be used. In another embodiment, the time for administration may be at the discretion of the human.

As described herein, one of the method embodiments is a method of feeding an animal or a human by administering to the animal or a human a feed or food composition or drinking water or a consumable liquid comprising an effective amount of an additive comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, wherein the Bacillus strain improves the health of the animal, particularly gastrointestinal health.

The improvement in health can be relative to an animal or a human not fed the bacterial strain. In one embodiment, Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, can increase the digestibility of a diet by producing enzymes that increase the digestibility of consumed nutrients where the enzymes are selected from the group consisting of an α-galactosidase, a protease, a phytase, a lipase, an amylase, a xylanase, a cellulase, a methionine reductase, a methionine synthase, a uricase, a prolyl endopeptidase, and combinations thereof. The enzyme can also be any other enzyme that degrades long chain fatty acids, such as enzymes that degrade stearic, palmitic, and/or oleic acid, but not limited to these fatty acids. Such an increase in digestibility of a diet leads to improvements in animal and human health.

In the embodiment where the effect is improving the health of the animal or the human, the improvement can result from a mechanism including, but not limited to, antimicrobial activity of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof. In various embodiments, the antimicrobial activity is against a microbe selected from the group consisting of E. coli, Salmonella, Staphylococcus, Enterococcus, Clostridia, Campylobacter, Candida, Mucor, Penicillium, Aspergillus, and combinations thereof. Thus, Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof can improve gut health of the animal or the human, and reduce pathogens in the animal or the human, and in the animal's environment. In yet another embodiment, Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, can reduce the bioburden in an animal or a human or increase the immune response in an animal or a human to improve the health of the animal or the human. The improvements described herein can be relative to an animal or a human not fed the bacterial strain.

In this method embodiment, the method can improve the health of the animal by improving the animal's environment by effects selected from the group consisting of reducing respiratory problems of the animal, improving gut health of the animal, improving consistency of performance of the animal, reducing diseases related to environmental toxicity in the animal, and reducing pathogens in the animal. In an embodiment where the animal is a poultry species, the method can improve the health of the animal by an effect selected from the group consisting of reducing respiratory problems of the poultry species, reducing breast blisters of the poultry species, improving consistency of performance of the poultry species, and reducing damage to the feet of the poultry species. These mechanisms of improvement to the health of the animal are non-limiting examples.

In additional embodiments of the invention, compositions comprising Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, are provided. In one embodiment, a commercial package is provided comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof.

In another embodiment, a feed additive for an animal feed or human food is provided comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof.

In yet another embodiment, an additive for the drinking water of an animal or a human or for a consumable liquid for a human is provided comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof.

In yet another illustrative aspect of the invention, an animal feed composition or a human food composition is provided comprising an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof.

In one embodiment, the feed additive for addition to an animal feed blend to produce a complete feed composition or for drinking water or the additive for addition to human food, drinking water, or a consumable liquid can be mixed with the animal feed blend or drinking water, or with a human food, drinking water, or a consumable liquid for example, with an automated micro-nutrient delivery system, or, for example, by hand-weighing and addition to achieve any of the doses of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, described herein, for administration to the animal or the human in the form of a complete feed or human food composition or drinking water or a consumable liquid. The mixing can also be done by any other suitable method known in the art for combining direct-fed microbials or probiotics with an animal feed blend or with human food or with drinking water or a consumable liquid to obtain a uniform mixture. In various embodiments, the mixing can be done for any suitable time period (e.g., about 1 to about 4 minutes).

In the embodiment where Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, are in the form of an additive for the drinking water of the animal or the human, or for a consumable liquid, the Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, can be in the form of, for example, a powder, a liquid, a gel, a freeze-dried form, a top-dressing, or pellets, and can be mixed with the drinking water or consumable liquid using any suitable method known in the art to achieve any of the doses of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, described herein, for administration to the animal or the human in the drinking water or consumable liquid for the animal or the human.

Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, can also be fed directly to the animal or the human orally (i.e., by oral insertion) in the form of a powder, a liquid, a gel, a freeze-dried form, a top-dressing, a capsule, a tablet, granules, a spray, a paste, a liquid drench, or a pellet.

In any of the composition embodiments described herein, the Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, can cause an improvement in the health of the animal or the human, particularly gastrointestinal health. The commercial package, feed or food additive, feed or food composition, or additive for the drinking water or for a consumable liquid for the animal or the human described herein can also inhibit a pathogen selected from the group consisting of E. coli, Salmonella, Staphylococcus, Enterococcus, Clostridia, Campylobacter, Candida, Mucor, Penicillium, Aspergillus and combinations thereof. These effects are non-limiting examples of the types of effects Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, can cause.

In one illustrative aspect, the feed or food additive, the additive for the drinking water or a consumable liquid for the animal or the human, or the feed or food composition can be in the form of a commercial package, such as a dietary nutrient composition (e.g., a probiotic composition or a direct-fed microbial composition). In another illustrative embodiment, the feed or food additive or additive for the drinking water or a consumable liquid for the animal or the human, or the Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, in the commercial package can be in the form of a concentrate (e.g., about 1×10⁸ to about 5×10⁹ CFU/g) or a superconcentrate (e.g., about 1×10¹⁰ to about 5×10¹² CFU/g). In another embodiment, the feed or food additive, feed or food composition, or additive for the drinking water or a consumable liquid for the animal or the human, or the Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, in a composition in a commercial package, can be in a dry form (e.g., a powder), a pelleted form, a liquid form, a freeze-dried form, in the form of a top-dressing, a paste, a liquid drench, or in the form of a gel, a capsule, a tablet, a spray, granules, or any other suitable form.

In another illustrative embodiment, the commercial package, feed or food additive, additive for the drinking water or a consumable liquid for the animal or the human, or feed or food composition can further comprise a carrier for the Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof. The carrier can be selected from the group consisting of a bran, rice hulls, a salt, mineral oil, a dextrin (e.g., maltodextrin), whey, sugar, limestone, dried starch, sodium silico aluminate, vegetable oil, inulin, and combinations thereof. In another embodiment, the carrier can be any suitable carrier known in the art for a direct-fed microbial or a probiotic composition. The carrier is exogenously added to the bacterial strain (i.e., not naturally present or not present in nature with the bacterial strain). In another embodiment, the commercial package, feed or food additive, additive for the drinking water or consumable liquid for the animal or the human, or feed or food composition can further comprise a binder such as clay, yeast cell wall components, aluminum silicate, glucan, bentonites, zeolites, hydrated sodium calcium aluminosilicate, charcoal, chlorella, and sodium metabisulfite, or other known binders. The binder is exogenously added to the bacterial strain (i.e., not naturally present or not present in nature with the bacterial strain).

In any of the method or composition embodiments described herein, a prebiotic can also be added to the final feed composition, to the feed additive or the drinking water, or can be added separately. Exemplary prebiotics include, but are not limited to, non-digestible carbohydrates, fructo-oligosaccharides (FOS), galacto-oligosaccharides (GOS), trans-galacto-oligosaccharides, short and long chain fructans (e.g., FOS and inulin) and lactulose, oligosaccharide carbohydrates (OSCs), fructo-oligosaccharides or oligofructose, galacto-oligosaccharides (e.g., the GOS with excess galactose at C3, C4 or C6 and the GOS manufactured from lactose through enzymatic trans-glycosylation to provide, for example, a mixture of tri- to pentasaccharides with galactose in β(1→6), β(1→3), and β(1→4) linkages (i.e., trans-galacto-oligosaccharides or TOS)), resistant starch, glucose-derived oligosaccharides (e.g., polydextrose), pectic oligosaccharides (POS), and non-carbohydrate oligosaccharides (e.g., cocoa-derived flavanols).

In yet other embodiments, the commercial package, feed or food additive, additive for the drinking water or a consumable liquid for the animal or the human, or feed or food composition comprising Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, is in a container for commercial use. In various embodiments the container can be, for example, a bag (e.g., a 20-pound bag, a 50-pound bag, a 2-ounce bag, a 1-pound bag, or a 1-kilogram bag), a pouch, a drum, a bottle, or a box. In illustrative aspects, the container for the commercial package, feed or food additive, additive for the drinking water or consumable liquid for the animal or the human, or feed or food composition comprising Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), and/or a strain having all of the identifying characteristics of Bacillus strain (NRRL No. B-68054), or combinations thereof, can comprise plastic, metal, foil, paper, fiber, or cardboard (e.g., a plastic pail, a paper bag, a foil bag, a fiber drum, etc.). The commercial package, feed or food additive, additive for the drinking water or consumable liquid for the animal or the human, or feed or food composition can further comprise instructions for use of one or more of the Bacillus strains.

In one aspect, the commercial package, feed or food additive, additive for the drinking water or consumable liquid for the animal or the human, or feed or food composition described herein can further comprise an exogenously added nutrient component (i.e., a nutrient component not present with the bacterial strain in nature) selected from the group consisting of a vitamin, an antibiotic, an enzyme, a water-soluble or water-insoluble monosaccharide, disaccharide, or polysaccharide, a fat, phosphorous, sodium bicarbonate, limestone, calcium, sodium, sulfur, magnesium, potassium, copper, iron, manganese, zinc, fish oil, raw seed, an antioxidant, and a starch.

In one embodiment, the exogenously added nutrient component is an enzyme and the enzyme is selected from the group consisting of a galactosidase, a protease, a lipase, an amylase, a hemicellulase, an arabinoxylanase, a xylanase, a cellulase, an NSPase, a phytase, a methionine reductase, a methionine synthase, a uricase, a prolyl endopeptidase, and combinations thereof.

In any of the embodiments described herein, the Bacillus strain may not cause hemolysis, may not cause cytotoxicity, may have antioxidant activity, and/or may have quorum quenching activity. In any of the embodiments described herein, the Bacillus strain may not produce a toxin selected from the group consisting of hemolysin BL subunit A (hb1A), hemolysin BL subunit C (hb1C), hemolysin BL subunit D (hb1D), nonhemolytic enterotoxin subunit A (nheA), nonhemolytic enterotoxin subunit B (nheB), nonhemolytic enterotoxin subunit C (nheC), emetic toxin, enterotoxin FM (entFM), enterotoxin T (bceT), and cytotoxin K (cytK). In any of the embodiments described herein, the Bacillus strain may produce an anti-inflammatory biomarker selected from the group consisting of SOCS1, TOLLIP, IL-10, and CXCL12.

The following examples are for illustrative purposes only. The examples are non-limiting, and are not intended to limit the invention in any way.

Bacillus Strain BC1 (NRRL No. B-67744)

### Example 1

### Antibiotic Resistance

Resistance to therapeutic antibiotics by microbial pathogens is currently considered one of the greatest challenges in medicine and public health, as some infectious diseases may become virtually untreatable if they become non-respondent to current therapies. Antibiotic resistance may be classified into two types; intrinsic/natural or extrinsic/acquired. Intrinsic/natural is when resistance is inherent to a bacterial species, and is a trait generally shared by all members of that species. Extrinsic/acquired is when a strain of a typically susceptible species is resistant to a given antimicrobial drug. Extrinsic/acquired resistance can occur either from the gain of exogenous DNA or mutation of indigenous genes. While intrinsic resistance likely presents a very low risk of dissemination, extrinsic resistance, especially when the relevant genes are associated with mobile genetic elements such as plasmids and transposons, can be transferred to pathogens or other commensal bacteria. It is generally recommended that resistance to antibiotics be assessed in all probiotic strains prior to marketing. Phenotypic evaluation of antibiotic resistance involves testing the capacity of a microorganism to survive in a medium containing different concentrations of antibiotics. Whereas most microorganisms can survive at low concentrations of many antibiotics, resistance is defined as the capacity to grow at antibiotic concentrations similar to those reached in the human body during therapeutic intervention.

The inventors have assessed resistance phenotypically. A bacterial strain is defined by EFSA as susceptible when its growth is inhibited at concentration of a specific antibiotic that is equal to or lower than the established cut-off value for that particular species. A bacterial strain is defined as resistant when it is able to grow at a concentration of a specific antibiotic that is higher than the established cut-off. Both the EFSA and CLSI guidelines for determining susceptibility and resistance were interpreted through their defined cut-off values. See Table 1.

TABLE 1

| | NRRL | EFSA | NRRL No. | CLSI | NRRL No. |
|---|---|---|---|---|---|
| | No. B- | Breakpoints | B-67744 | Breakpoints | B-67744 |
| | 67744 | for | EFSA | for | CLSI |
| | ug/ml | Bacillus | Interpre- | Staphylococcus | Interpre- |
| Antibiotic | (triplicate) | spp. | tation | spp. | tation |
| Chloramphenicol | 0.016 | 8 | Sensitive | ≤8 | Sensitive |
| Clindamycin | 0.016 | 4 | Sensitive | ≤0.5 | Sensitive |
| Ciprofloxacin | 0.064 | n.r. | n.r. | ≤1 | Sensitive |
| Erythromycin | 0.125 | 4 | Sensitive | ≤0.5 | Sensitive |
| Gentamicin | 0.023 | 4 | Sensitive | ≤4 | Sensitive |
| Kanamycin | 0.016 | 8 | Sensitive | ≤16 | Sensitive |
| Linezolid | 0.75 | n.r. | n.r. | ≤4 | Sensitive |
| Rifampin | 0.25 | n.r. | n.r. | ≤1 | Sensitive |
| Streptomycin | 1.5 | 8 | Sensitive | n.r. | n.r. |
| Tetracycline | 1.25 | 8 | Sensitive | ≤4 | Sensitive |
| Tigecycline | 0.064 | n.r. | n.r. | ≤0.5 | Sensitive |
| Vancomycin | 0.38 | 4 | Sensitive | ≤2 | Sensitive |

### Example 2

### Hemolysis Activity

Bacillus strain BC1 (NRRL No. B-67744) was tested for hemolysis activity by methods using blood agar plates to determine zero, partial, or complete lysis of the red blood cells. This strain had zero hemolysis activity. Beta-hemolysis is defined as complete or true lysis of red blood cells. A clear zone, approaching the color and transparency of the base medium, surrounds the colony. Alpha-hemolysis (or partial hemolysis) is the reduction of the red blood cell hemoglobin to methemoglobin the medium surrounding the colony. Discoloration resulting in brown or green colored cells surrounding the medium is common of alpha hemolysis. Unlike beta hemolysis, alpha hemolysis maintains the structure of the cell membrane. This strain was cultured and tested for antibiotic susceptibility by the following methods. First, the strain was cultured from a frozen stock by using a sterile inoculating loop to iso-streak the cells on MRS media and incubated anaerobically at 45° C. for 24 hours. When the colonies were visible the next day, they were morphologically examined for purity and a sterile inoculating loop was used to collect a single colony. The single colony was cultured to a higher density by inoculating it into BHI Broth+yeast extract+cysteine and incubated anaerobically at 45° C., 24 hours. The next day, the growth culture was standardized to a cell density of OD_{600 nm} 0.5 using 0.1% peptone as a diluent. The blood agar plates were purchased pre-made and comprised of tryptic soy agar with 5% sheep's blood. Using a sterile inoculating loop with a diameter that holds approximately 10 μl, the culture was streaked horizontally across the surface of the plates, in triplicate. The plates were then incubated aerobically at 45° C., 24 hours. The next day, the plates were removed from the incubator and results were recorded. Results were determined as ‘zero hemolysis’ for no discoloration of the media, ‘alpha hemolysis’ partial hemolysis and green-brown discoloration of the media, and ‘beta hemolysis’ for complete lysis of the media and display of clearing zone around the cells. This strain produced zero hemolysis around the cells streaked on the blood agar plate for all three triplicate streaks.

### Example 3

### Enterotoxins

Bacillus strain BC1 (NRRL No. B-67744) testing resulted in negative presence for all known toxin genes commonly associated with Bacillus cereus and Bacillus thuringiensis using a Multiplex PCR method adapted from Yang et al. Whole genome sequence analysis was also used to query for these known Bacillus toxin gene sequences and the database yielded no significant similarities found. This strain (NRRL No. B-67744) was cultured, DNA was extracted, and the DNA was tested for toxins by the following methods. First, the strain was cultured from a frozen stock by using a sterile inoculating loop to iso-streak the cells on MRS media and incubated aerobically at 45° C., 24 hours. When the colonies were visible the next day, they were morphologically examined for purity and a sterile inoculating loop was used to collect a single colony to extract DNA. The single colony was cultured to a higher density by inoculating it into BHI+yeast extract+cysteine broth and incubated anaerobically at 45° C. for 24 hours. The next day, 1 ml of the culture was transferred to a 1.5 ml microcentrifuge tube, and the cells were pelleted in a centrifuge at 10,000 rpm for 10 minutes. DNA extraction of those cells was carried out using Qiagen's DNeasy Blood and Tissue single column kit. The supernatant from the pelleted cells was discarded and the pellet was resuspended in 180 μl of lysis buffer containing 20 mg/ml of lysozyme. NRRL No. B-67744+lysis solution was incubated at 37° C. for 45 minutes to break apart the cell walls and lyse the cellular components. Then, 20 μl proteinase K and 200 μl Buffer AL were added and the sample was incubated for an additional 30 minutes at 56° C. to degrade proteinaceous components. Upon completion of incubation, 200 μl EtOH was added and vortexed to a homogenous solution to aggregate insoluble DNA. Spin columns were used according to the manufacturer's recommended protocol to separate and clean DNA from the solution. The DNA was eluted in 100 μl Low-TE Buffer purchased from ThermoFisher.

The primer sequences and multiplex PCR methods were adapted from Yang et al. Primers for toxins Hemolysin BL subunit A (hb1A), Hemolysin BL subunit C (hb1C), Hemolysin BL subunit D (hb1D), Nonhemolytic enterotoxin subunit A (nheA), Nonhemolytic enterotoxin subunit B (nheB), Nonhemolytic enterotoxin subunit C (nheC), Emetic toxin, Enterotoxin FM (entFM), Enterotoxin T (bceT), and Cytotoxin K (cytK) were used to test the presence of Bacillus spp. producing toxins. These primer sequences were purchased from Eurofins and diluted to working concentrations of 40-350 nM (Table 2). Three separate master mixes were prepared for the multiplex toxin assays as seen in Table 1. For each master mix, the total volume was 49 μl, comprising of 200 μM DNTPs, 1×PCR Buffer, 2U fast start Taq polymerase, and the remaining volume with ddH₂O. The total reaction volume was 55 μl, comprising of 49 μl respective master mix and 6 μl of template DNA. Bacillus cereus and Bacillus thuringiensis genomic DNA were used as positive controls, and ddH₂O was used for no template controls.

The PCR reaction was performed on an Applied Biosystems 2720 Thermal Cycler utilizing endpoint PCR technologies. All three master mixes were tested against the NRRL 67744, Bc positive control, Bt positive control, and NT negative control. The reaction conditions consisted of a denaturation step at 95° C. for 5 min followed by 30 cycles of 95° C. for 30 seconds, 60° C. for 30 seconds, 72° C. for 45 seconds, and finished with an elongation step at 72° C. for 7 minutes. The amplification products were analyzed on a 2% agarose gel, with electrophoresis for 80 minutes at 100 volts, and visualized using a Gel Imaging System with UV transilluminator and GeneSnap software tools.

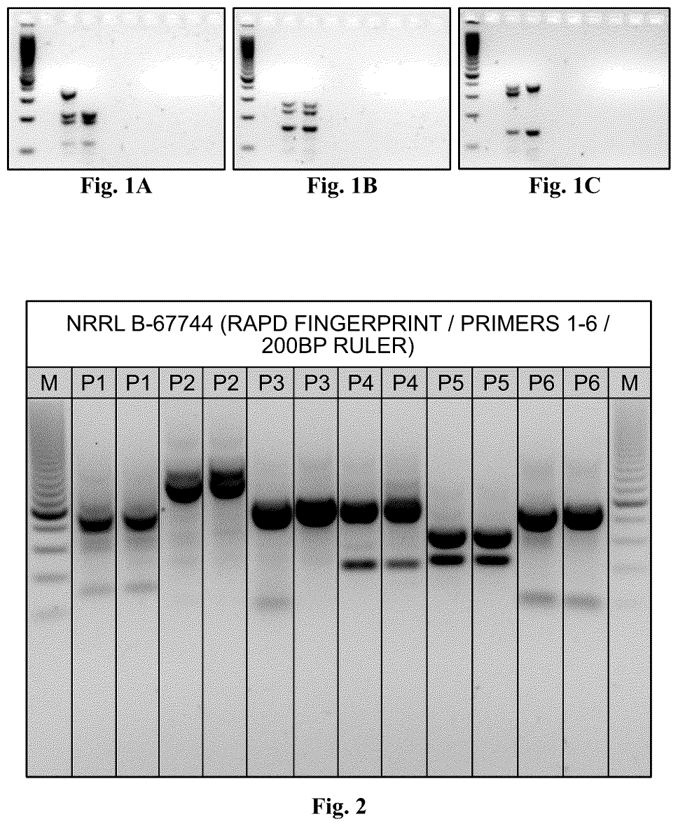

NRRL No. B-67744 genomic DNA was negative for the presence of any toxins within the three multiplex assays through 30 cycles of amplification as shown in FIG. 1. The genomic DNA controls for Bc and Bt yielded amplification products at the anticipated band sizes for each of the of the multiplexed assays, and the no template control was negative in all three master mixes.

Results of whole genome sequence analysis yielded no significant similarity of NRRL No. B-67744 with gene sequences for these known Bacillus cereus and Bacillus thuringiensis toxins (Table 3). The multiplex PCR assay yielded no amplification for Bacillus cereus-like toxin genes Hemolysin BL, Nonhemolytic Enterotoxin, Enterotoxin FM, Enterotoxin T, nor Cytotoxin K for NRRL No. B-67744 DNA. Amplified PCR products were seen at expected band sizes and additionally there were no stray bands at unexpected sizes for the genomic DNA positive controls, all with no amplification was seen in the no template control. Furthermore, as stated in Yang et al., the sensitivity of the multiplex PCR assay was approximately 100 pg and the primers were specific to the designed target. All of this data suggests that these primer sequences, paired in multiplex reactions, are an adequate method of analysis. These results were confirmed by blasting the whole genome sequence against the toxin gene sequencing via BLASTn database, displayed in Table 3.

TABLE 2
Master Mix Groups and Primer Concentrations for Each Primer Set

| | | Primer | | Concentration | Fragments |
|---|---|---|---|---|---|
| Set | Targeted gene | name | Sequence (5′ to 3′) | used (nM) | size (BP) |
| 1 | Hemolysin BL | HA-F1 | ATT AAT ACA GGG GAT GGA GAA | 150 | 237 |
| | subunit A | | ACT T (SEQ ID NO: 1) | | |
| | (hbLA) | HA-RI | TGA TCC TAA TAC TTC TTC TAG | | |
| | | | ACG CTT (SEQ ID NO: 2) | | |
| | Hemolysin BL | HC-F1 | CCT ATC AAT ACT CTC GCA ACA | 240 | 386 |
| | subunit C | | CCA AT (SEQ ID NO: 3) | | |
| | (hbLC) | HC-RI | TTT TCT TGA TTC GTC ATA GCC | | |
| | | | ATT TCT (SEQ ID NO: 4) | | |
| | Hemolysin BL | HD-F1 | AGA TGC TAC AAG ACT TCA AAG | 240 | 436 |
| | subunit D | | GGA AAC TAT (SEQ ID NO: 5) | | |
| | (hbLD) | HD-A1 | TGA TTA GCA CGA TCT GCT TTC | | |
| | | | ATA CTT (SEQ ID NO: 6) | | |
| | Emetic strain- | EM-F1 | AGC TTG GAG CGG AGC AGA CTA | 500 | 635 |
| | specific | | TGT (SEQ ID NO; 7) | | |
| | sequence | EM-RI | GTA TTT CTT TCC CGC TTG CCT TTT | | |
| | | | (SEQ ID NO: 8) | | |
| 2 | Nonhemolytlc | NA-F1 | ATT ACA GGG TTA TTG GTT ACA | 150 | 475 |
| | enterotoxin | | GCA GT (SEQ ID NO: 9) | | |
| | subunit A(nheA) | NA-RI | AAT CTT GCT CCA TAC TCT CTT | | |
| | | | GGA TGC T (SEQ ID NO: 10) | | |
| | Nonhemolytlc | NB-F1 | GTG CAG CAG CTG TAG GCG GT | 180 | 328 |
| | enterotoxin | | (SEQ ID NO: 11) | | |
| | subunit B(nheB) | NB-R1 | ATG TTT TTC CAG CTA TCT TTC | | |
| | | | GCA AT (SEQ ID NO: 12) | | |
| | Nonhemolytlc | NC-F1 | GCG GAT ATT GTA AAG AAT CAA | 450 | 557 |
| | enterotoxin | | AATGAG GT (SEQ ID NO: 13) | | |
| | subunit B(nheC) | NC-RI | TTT CCA GCT ATC TTT CGC TGT | | |
| | | | ATG TAA AT (SEQ ID NO: 14) | | |
| 3 | Enterotoxin FM | Fm-F1 | CAA AGA CTT CGT AAC AAA AGG | 160 | 290 |
| | (entFM) | | TGG T (SEQ D NO: 15) | | |
| | | Fm-R1 | TGT TTA CTC CGC CTT TTA CAA | | |
| | | | ACT T (SEQ ID NO: 16) | | |
| | Enterotoxin | Bcet-F1 | AGC TTG GAG CGG AGC AGA CTA | 700 | 701 |
| | T(bceT) | | TGT (SEQ ID NO: 17) | | |
| | | Bcet-RI | GTA TTT CTT TCC CGC TTG | | |
| | | | CCT TTT (SEQ ID NO: 18) | | |
| | Cytotoxln K(cytK) | Cyt-F1 | ATC GGG CAA AAT GCA AAA ACA | 500 | 800 |
| | | | CAT (SEQ ID NO: 19) | | |
| | | Cyt-RI | ACC CAG TTT GCA GTT CCG AAT GT | | |
| | | | (SEQ ID NO: 20) | | |
| | Cytotoxln K(cytK) | Cyt-F2 | ATC GGT CAA AAT GCA AAA ACA | 500 | 800 |
| | | | CAT (SEQ ID NO: 21) | | |
| | | Cyt-R2 | ACC CAG TTA CCA GTT CCG AAT | | |
| | | | GT (SEQ ID NO: 22) | | |

TABLE 3
Results from Whole Genome Sequencing
Analysis of Bc and Bt Toxin Genes

| | GenBank | Query Result |
|---|---|---|
| Toxin Gene | Accesion # | for NRRL 67744 |
| hbl | U63928.1 | No significant |
| (subunits C/D) | | similarity found |
| nhe | DQ885236.1 | No significant |
| (subunits A/B/C) | | similarity found |
| cytK | AJ277962.1 | No significant |
| | | similarity found |
| entFM | AY789084.1 | No significant |
| | | similarity found |
| bceT | D17312.1 | No significant |
| | | similarity found |

### Example 4

### Cytoxicity Assays

EFSA requires Bacillus spp. be free of toxigenic activity so an in vitro cytotoxicity test on B. coagulans NRRL No. B-67744, using Vero (epithelial) cells was conducted. Cytotoxicity was measured with a lactate dehydrogenase (LDH) assay and was done in accordance with EFSA's “Guidance on the characterization of microogranisms used as feed additives or as production organisms”. Absorbance values above 20% of the absorbance obtained from the maximum LDH release control (Promega 10× Lysis Solution) indicates cytotoxicity. The supernatant from B. coagulans NRRL No. B-67744, at 10% concentration as shown in Tables 4 and 5 below, had no cytotoxic effect against Vero cells.

TABLE 4
Bacillus coagulans NRRL No. B-67744 Percent LDH
Released by Vero Cells after 60 minutes exposure

| | Repli- | Repli- | Repli- | |
|---|---|---|---|---|
| Test Article | cate #1 | cate #2 | cate #3 | Average |
| B. coagulans | 1.31% | 1.47% | 1.63% | 1.47% |
| NRRL No. | | | | |
| B-67744% | | | | |
| LDH release | | | | |
| Untreated Control | 1.41% | 0.66% | 1.09% | 1.06% |

As a control for endogenous LDH signal in the bacterial medium used to generate the test article, the test article was also tested in EC buffer (in the absence of Vero cells). The results of this assay are in the table above, as denoted in the equation below the table.

TABLE 5
Bacillus coagulans NRRL No. B-67744 Percent LDH
Signal in EC Buffer and Supernatant Controls

| | Repli- | Repli- | Repli- | |
|---|---|---|---|---|
| Test Article | cate #1 | cate #2 | cate #3 | Average |
| B. coagulans | 0.61% | 0.61% | 0.56% | 0.59% |
| NRRL No. | | | | |
| B-67744% | | | | |
| LDH release | | | | |

### Example 5

### RAPD-PCR DNA Profiles

The randomly amplified polymorphic DNA PCR method (RAPD-PCR) was used to identify genetic variability of the strain. Preparation of the DNA to be used in the RAPD-PCR reaction was performed using Qiagen's Blood and Tissue single column kit. To obtain DNA, an overnight culture was prepared, struck for purity, pelleted, and DNA was extracted following the manufacturer's protocol. Preparation of the RAPD-PCR reactions was done by using a Cytiva RAPD bead kit, which entails using one bead per reaction, sterile water, and DNA template, along with one of six primers pre-designed to randomly amplify the polymorphic DNA. All six primers were used in separate reactions for each strain. Each 25 ul reaction contained 1 Cytiva RAPD bead, 16 ul sterile water, 5 ul respective primer, and 4 ul DNA template. Each sample was sealed, vortexed, and centrifuged briefly prior to running the reaction. The RAPD-PCR reaction was performed with the following run conditions in an AB2720 thermocycler; 95° C. 5 min, followed by 45 cycles of (95° C. 1 min, 36° C. 1 min, 72° C. 2 min), followed by 72° C. 7 min, and finished with a 4° C. indefinite hold to preserve the product. The RAPD-PCR product was analyzed by gel electrophoresis using 1% (wt/vol) agarose in 1×TBE buffer and a UV imager. The agarose gel contained SyberSafe Gel Stain at 1 ul/10 ml. The gel ran at 120 volts for 3 hours. At the end of the run, a digital camera attached to a UV imager uploaded the gel image to GeneSnap software which subsequently inverted the saturation values and adjusted the contrast for viewing purposes. See FIG. 2.

### Example 6

### Digestive Enyzme Production

Phenotypic enzyme assays using NRRL 67744 resulted in positive enzymatic activity for lipase, amylase, protease, and cellulase, and genomic analysis further yielded genomic potential for alpha and beta galactosidases, and methionine reductases and synthases. These enzymes are extraordinary biocatalysts that increase the rate of biochemical reactions, particularly during digestion. The enzymes and reactions are responsible for hydrolyzing complex carbohydrates, lipids, and proteases down into more bioavailable components, thus increasing the rate of absorption and nutrient intake.

The phenotypic enzyme activity plate assays were performed to determine the enzymatic potential for digestive enzymes well-known to be produced by resident microbiota. Tryptic Soy Agar (TSA) was the base agar media used along with a selective agent specific to each of the enzymes. For lipase activity, 50 mL Tween 80 and 2.5 mL Polysorbate 80 were added to 1 L TSA. For Amylase activity, 10 g of corn starch was added to 1 L TSA. For protease activity, 10 g of Casein was added to 1 L TSA. For cellulase activity, 5 g of CMC was added to 1 L TSA. All medium was autoclaved at 122° C. for 30 min and then poured and dried into agar plates. For each set of plates, 10 ul of NRRL 67744 was spotted in triplicate, and incubated anaerobically at 37° C., 24 hours. The next day, positive enzymatic activity was determined by a zone of clearing around the culture spot. A negative reaction was indicated if there was no zone of clearing.

### Example 7

### Immunomodulation

Bacillus species are known to modulate immunity through interactions with the host gastrointestinal tract. This immune modulation can help the human body keep a delicate balance between eliminating invading pathogens, while still maintaining a regulated level of inflammatory response capable of returning to homeostasis. Research has uncovered that the 70% of a human's immune system is localized within the epithelial tissues of the gastrointestinal tract. Bacillus, and particularly NRRL No. B-67744, are capable of communicating directly and indirectly with these immune cells in order to provide a necessary response to the surrounding environment, providing essential health benefits to the host, and regulating immune homeostasis.

The rat IEC-6 cell line was developed from rodent intestinal epithelial cells, which form numerous microvilli and features of typical crypt cells, with tight junctions linking adjacent cells. Rat IEC-6 cells represent a well characterized model to study the intestinal epithelial response to bacterial infection. This cell line expresses the features of intestinal cells and mediates a response from many different immune cell types. For testing the immunomodulation capacity of NRRL No. B-67744, the strain was exposed to IEC-6 cells in the presence and absence of LPS cell wall component. IEC-6 cells were cultured and passaged twice for consistency of viability using DMEM cell culture media supplemented with 10% FBS and 1% antibiotic/antimycotic and incubated at 37° C. with 5% CO₂. For the immune assay, cells were cultured in 24-wells plates, with 500 ul volumes, and seeded at a density of 100 k/well. A confluent monolayer was maintained for 2 days prior to exposure to allow for full maturation of the immune cells within the mucosal epithelial monolayer. Once maturity is reached, the antibiotic/antimycotic is removed from the media and given a four-hour equilibrium period.

To prep the NRRL No. B-67744 test article, an overnight culture was grown in 5 ml BHI+yeast extract+cysteine and incubated at 42° C. The culture was quantitated using optical density measurements (OD at 600 nm), using an OD of 0.5 as the reference for 10⁷ cell counts for Bacillus. The sample was diluted using 1 ml sterile PBS to create 10⁶, 10⁵, 10⁴, and 10³ concentrations. The culture was also counted for exact cell counts. To prep the LPS compound, a thawed an aliquot of 100 ng/ul solution and diluted that down to 10 ng/ul using PBS. For the immune assay, 5 ul of 10 ng/ul LPS was used as the stimulation agent, and 10 ul of culture was used for each dose of the NRRL No. B-67744 strain, as pictured below. Each test condition and control had six replicates. Plates were incubated for 2 hrs at 37° C., 5% CO₂.

The cells were harvested after the two-hour incubation period, and RNA was extracted for expression analysis. Briefly, working with one well at a time as to go quickly and not allow the expression to change, media was removed and 200 ul of Tri reagent was added. The Tri reagent was allowed to work at room temperature for 5 minutes, then once the cells were released from being bound to the plate well, they were removed from the plate and placed into a 96-well 2 ml round bottom block. Block was covered with breathable sterile film at the first layer and an adhesive foil at the second layer. The block was then snap frozen in liquid nitrogen and stored at −80° C. until ready for RNA extraction and expression work.

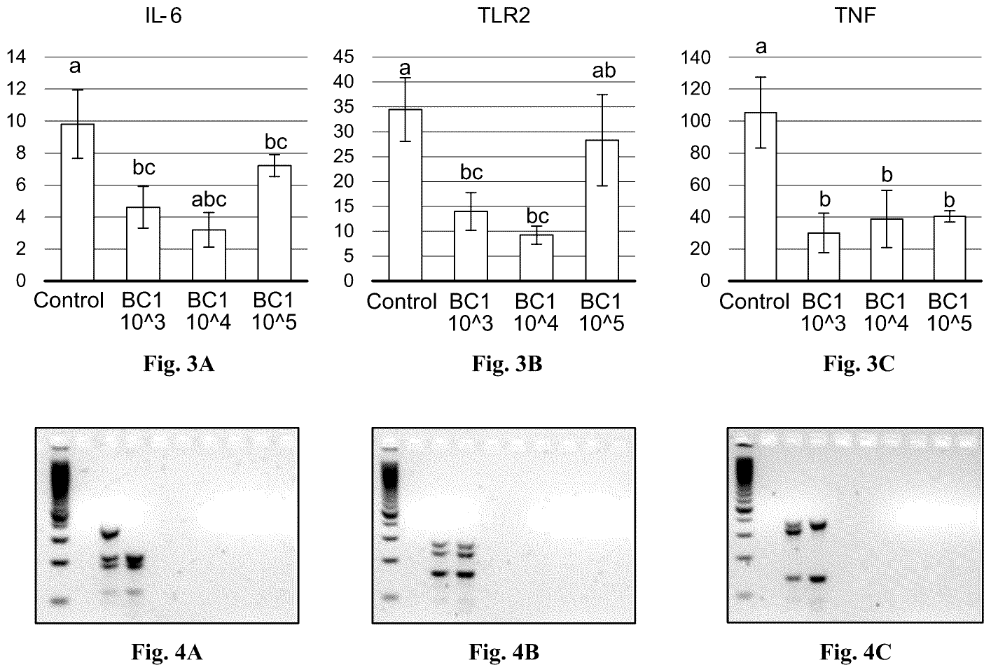

RNA extraction was completed using Qiagen's RNeasy 96-well kit, using DTT added to the RLT buffer. RLT+DTT buffer was prepared by adding 40 ul/mL of 1M DTT into RLT buffer. The manufacturers protocol was followed, and two rounds of 45 uL was eluted. The RNA was stabilized into cDNA immediately after extraction using QuantaBio reverse transcriptase. For the reaction, 16 ul of RNA and 4 ul of rt-enzyme was added to a 96-well PCR plate. The reaction was ran on a Bio-Rad machine using a standard reverse transcription protocol provided by QuantaBio. For the immunomodulation assays, immune biomarker expression was quantitated and expressed as average relative quantity (RQ) values. GAPDH was used as a reference housekeeping gene. The results in FIG. 3 below illustrate the average RQ value difference when compared to the LPS control.

Results of the immune response from IEC-6 cells show a significant difference in biomarker expression of the pro-inflammatory cytokines and pathogen receptor when compared to an LPS stimulant (IL-6, TLR2, and TNFα). This data suggests that strain NRRL No. B-67744 has anti-inflammatory affects after a stimulation event occurs.

### Example 8

### Antioxidant Activity

Genomic analysis yielded potential for numerous natural enzymatic antioxidants, both primary (Superoxide dismutase genes for Manganese, Catalase activity, and Glutathione peroxidase) and secondary (glucose-6-phosphate-dehydrogenase), as well as inhibition of protein oxidation through Thioredoxin and Glutaredoxin activity. While oxygen is a necessary element to many functions of living organisms, oxygen concentrations that exceed the normal range causes oxidative stress through the generation of reactive oxygen species (ROS). ROS is naturally produced through everyday occurrences both endogenously and exogenously. Endogenous sources include byproducts of metabolic processes, NADPH oxidases, mitochondrial electron transport chain leakage, and cytokine and growth factor receptors; while exogenous sources come from UV light, radiation, drugs, pollutants, and/or pathogens. The resulting oxidative stress can cause destructive damage on DNA/RNA, proteins, and lipids, and also result in cellular responses such as inflammation and carcinogenesis. If excessive amounts of ROS are not controlled, these destructive changes often lead to chronic diseases including atherosclerosis, arthritis, diabetes, Alzheimer's disease, neurodegenerative diseases, and cardiovascular diseases.

Antioxidants are ROS scavengers that can shield, scavenge, and repair oxidative damage, thereby defending target assemblies or molecules from oxidative damages. Living organisms have enzymatic and nonenzymatic antioxidant mechanisms for inactivating ROS, and microbes have been identified as a source for both. Enzymes, including catalase, glutathione peroxidase, and superoxide dismutase, are the endogenous antioxidants that control ROS damage, whereas carotenes, flavonoids, coenzyme Q, vitamins, minerals, and phenolic acids are the sources of exogenous antioxidants.

Bacillus Strain (NRRL No. B-68053)

### Example 9

### Antibiotic Resistance

The inventors have assessed resistance phenotypically. A bacterial strain is defined by EFSA as susceptible when its growth is inhibited at concentration of a specific antibiotic that is equal to or lower than the established cut-off value for that particular species. A bacterial strain is defined as resistant when it is able to grow at a concentration of a specific antibiotic that is higher than the established cut-off. We utilized both the EFSA and CLSI guidelines for determining susceptibility and resistance interpreted through their defined cut-off values. See Table 6.

TABLE 6
Results of Minimum Inhibitory Concentrations (MICs) Against Medical Antibiotics

| | NRRL | EFSA | NRRL No. | CLSI | NRRL No. |
|---|---|---|---|---|---|
| | No. B- | Breakpoints | B-68053 | Breakpoints | B-68053 |
| | 68053 | for | EFSA | for | CLSI |
| | ug/mL | Bacillus | Interpre- | Staphylococcus | Interpre- |
| Antibiotic | (triplicate) | spp. | tation | spp. | tation |
| Chloramphenicol | 3 | 8 | Sensitive | ≤8 | Sensitive |
| Clindamycin | 1 | 4 | Sensitive | ≤0.5 | Intermediate |
| Ciprofloxacin | 0.94 | n.r. | n.r. | ≤1 | Sensitive |
| Erythromycin | 0.064 | 4 | Sensitive | ≤0.5 | Sensitive |
| Gentamicin | 0.094 | 4 | Sensitive | ≤4 | Sensitive |
| Kanamycin | 0.75 | 8 | Sensitive | ≤16 | Sensitive |
| Linezolid | 1 | n.r. | n.r. | ≤4 | Sensitive |
| Rifampin | 0.25 | n.r. | n.r. | ≤1 | Sensitive |
| Streptomycin | 8 | 8 | Sensitive | n.r. | n.r. |
| Tetracycline | 12 | 8 | Resistant | ≤4 | Intermediate |
| Tigecycline | 0.094 | n.r. | n.r. | ≤0.5 | Sensitive |
| Vancomycin | 0.5 | 4 | Sensitive | ≤2 | Sensitive |

### Example 10

### Hemolysis Activity

Bacillus strain (NRRL No. B-68053) was tested for hemolysis activity by methods of blood agar plates to determine zero, partial, or complete lysis of the red blood cells. NRRL 68053 was negative for hemolysis activity. Beta-hemolysis is defined as complete or true lysis of red blood cells. A clear zone, approaching the color and transparency of the base medium, surrounds the colony. Alpha-hemolysis is the reduction of the red blood cell hemoglobin to methemoglobin the medium surrounding the colony. Discoloration resulting in brown or green colored cells surrounding the medium is common of alpha hemolysis. Unlike beta hemolysis, alpha hemolysis maintains the structure of the cell membrane. NRRL 68053 was cultured and tested for antibiotic susceptibility by the following methods. First, the strain was cultured from a frozen stock by using a sterile inoculating loop to iso-streak the cells on tryptic soy agar media and incubated overnight at 37° C. When the colonies were visible the next day, they were morphologically examined for purity and a sterile inoculating loop was used to collect a single colony. The single colony was cultured to a higher density by inoculating it into tryptic soy broth and incubated overnight at 37° C., 230 rpm. The next day, the growth culture was standardized to a cell density of OD_{600 nm} 0.8 using 0.1% peptone as a diluent. The blood agar plates were purchased pre-made and comprised of tryptic soy agar with 5% sheep's blood. Using a sterile inoculating loop with a diameter that holds approximately 10 μl, the culture was streaked horizontally across the surface of plate, in triplicate. The plates were then incubated overnight at 37° C. The next day, the plates were removed from the incubator and results were recorded. Results were determined as ‘no hemolysis’ for no discoloration of the media, ‘alpha hemolysis’ partial hemolysis and green-brown discoloration of the media, and ‘beta hemolysis’ for complete lysis of the media and display of clearing zone around the cells. NRRL No. B-68053 produced no hemolysis around the cells streaked on the blood agar plate for all three triplicate streaks.

### Example 11

### Enterotoxins

Bacillus strain NRRL No. B-68053 tesing resulted in negative presence for all known toxin genes commonly associated with Bacillus cereus and Bacillus thuringiensis using a Multiplex PCR method adapted from Yang et al. Whole genome sequence analysis was also used to query for these known Bacillus toxin gene sequences and the database yielded no significant similarities found. NRRL No. B-68053 was cultured, DNA was extracted, and tested for toxins by the following methods. First, the strain was cultured from a frozen stock by using a sterile inoculating loop to iso-streak the cells on tryptic soy agar media and incubated overnight at 37° C. When the colonies were visible the next day, they were morphologically examined for purity and a sterile inoculating loop was used to collect a single colony to extract DNA from the strain. The single colony was cultured to a higher density by inoculating it into tryptic soy broth and incubated overnight at 37° C. The next day, 1 ml of the culture was transferred to a 1.5 ml microcentrifuge tube, and the cells were pelleted in a centrifuge at 10,000 rpm for 10 minutes. DNA extraction of those cells were carried using Qiagen's DNeasy Blood and Tissue single column kit. The supernatant from the pelleted cells was discarded and the pellet was resuspended in 180 μl of lysis buffer containing 20 mg/ml of lysozyme. NRRL No. B-68053+lysis solution was incubated at 37° C. for 45 minutes to break apart the cell walls and lyse the cellular components. Then, 20 μl proteinase K and 200 μl Buffer AL were added and sample was incubated for an additional 30 minutes at 56° C. to degrade proteinaceous components. Upon completion of incubation, 200 μl EtOH was added and vortexed to a homogenous solution to aggregate insoluble DNA. Spin columns were used according to the manufacturer's recommended protocol to separate and clean DNA from the solution. The DNA was eluted in 100 μl Low-TE Buffer purchased from ThermoFisher.

The primer sequences and multiplex PCR methods were adapted from Yang et al. Primers for toxins Hemolysin BL subunit A (hb1A), Hemolysin BL subunit C (hb1C), Hemolysin BL subunit D (hb1D), Nonhemolytic enterotoxin subunit A (nheA), Nonhemolytic enterotoxin subunit B (nheB), Nonhemolytic enterotoxin subunit C (nheC), Emetic toxin, Enterotoxin FM (entFM), Enterotoxin T (bceT), and Cytotoxin K (cytK) to test presence of Bacillus spp. producing toxins. These primer sequences were purchased from Eurofins and diluted to working concentrations 40-350 nM (Table 7). Three separate master mixes were prepared for the multiplex toxin assays as seen in Table 7. For each master mix, the total volume was 49 μl, comprising of 200 μM DNTPs, 1×PCR Buffer, 2U fast start Taq polymerase, and the remaining volume with ddH₂O. The total reaction volume was 55 μl, comprising 49 μl respective master mix and 6 μl of template DNA. Bacillus cereus and Bacillus thuringiensis genomic DNA were used as positive controls, and ddH₂O was used for no template controls.

The PCR reaction was performed on an Applied Biosystems 2720 Thermal Cycler utilizing endpoint PCR technologies. All three master mixes were tested against the NRRL No. B-68053, Bc positive control, Bt positive control, and NT negative control. The reaction conditions consisted of a denaturation step at 95° C. for 5 min followed by 30 cycles of 95° C. for 30 seconds, 60° C. for 30 seconds, 72° C. for 45 seconds, and finished with an elongation step at 72° C. for 7 minutes. The amplification products were analyzed on a 2% agarose gel, with electrophoresis for 80 minutes at 100 volts, and visualized using a Gel Imaging System with UV transilluminator and GeneSnap software tools.

NRRL No. B-68053 genomic DNA was negative for the presence of any toxins within the three multiplex assays through 30 cycles of amplification, shown in FIG. 4. The genomic DNA controls for Bc and Bt yielded amplification products at the anticipated band sizes for each of the of the multiplexed assays, and the no template control was negative in all three master mixes. Results of whole genome sequence analysis yielded no significant similarity of NRRL No. B-68053 with gene sequences for these known Bacillus cereus and Bacillus thuringiensis toxins (Table 8).

The multiplex PCR assay yielded no amplification for Bacillus cereus-like toxin genes Hemolysin BL, Nonhemolytic Enterotoxin, Enterotoxin FM, Enterotoxin T, nor Cytotoxin K for NRRL No. B-68053 DNA. Amplified PCR products were seen at expected band sizes and additionally there were no stray bands at unexpected sizes for the genomic DNA positive controls, all with no amplification was seen in the no template control. Furthermore, as stated in Yang et al., the sensitivity of the multiplex PCR assay was approximately 100 pg and the primers were specific to the designed target. All of this data suggests that these primer sequences, paired in multiplex reactions, are an adequate method of analysis. These results were confirmed by blasting the whole genome sequence against the toxin gene sequencing via BLASTn database, displayed in Table 8.

TABLE 7
Master Mix Groups and Primer Concentrations for Each Primer Set

| | | Primer | | Concentration | Fragments |
|---|---|---|---|---|---|
| Set | Targeted gene | name | Sequence (5′ to 3′) | used (nM) | size (BP) |
| 1 | Hemolysin BL | HA-F1 | ATT AAT ACA GGG GAT GGA GAA | 150 | 237 |
| | subunit A | | ACT T (SEQ ID NO: 1) | | |
| | (hbLA) | HA-RI | TGA TCC TAA TAC TTC TTC TAG | | |
| | | | ACG CTT (SEQ ID NO: 2) | | |
| | Hemolysin BL | HC-F1 | CCT ATC AAT ACT CTC GCA ACA | 240 | 386 |
| | subunit C | | CCA AT (SEQ ID NO: 3) | | |
| | (hbLC) | HC-RI | TTT TCT TGA TTC GTC ATA GCC | | |
| | | | ATT TCT (SEQ ID NO: 4) | | |
| | Hemolysin BL | HD-F1 | AGA TGC TAC AAG ACT TCA AAG | 240 | 436 |
| | subunit D | | GGA AAC TAT (SEQ ID NO: 5) | | |
| | (hbLD) | HD-A1 | TGA TTA GCA CGA TCT GCT TTC | | |
| | | | ATA CTT (SEQ ID NO: 6) | | |
| | Emetic strain- | EM-FI | AGC TTG GAG CGG AGC AGA CTA | 500 | 635 |
| | specific | | TGT (SEQ ID NO; 7) | | |
| | sequence | EM-RI | GTA TTT CTT TCC CGC TTG CCT TTT | | |
| | | | (SEQ ID NO: 8) | | |
| 2 | Nonhemolytlc | NA-F1 | ATT ACA GGG TTA TTG GTT ACA | 150 | 475 |
| | enterotoxin | | GCA GT (SEQ ID NO: 9) | | |
| | subunit A(nheA) | NA-R1 | AAT CTT GCT CCA TAC TCT CTT | | |
| | | | GGA TGC T (SEQ ID NO: 10) | | |
| | Nonhemolytlc | NB-F1 | GTG CAG CAG CTG TAG GCG GT | 180 | 328 |
| | enterotoxin | | (SEQ ID NO: 11) | | |
| | subunit B(nheB) | NB-R1 | ATG TTT TTC CAG CTA TCT TTC | | |
| | | | GCA AT (SEQ ID NO: 12) | | |
| | Nonhemolytlc | NC-F1 | GCG GAT ATT GTA AAG AAT CAA | 450 | 557 |
| | enterotoxin | | AATGAG GT (SEQ ID NO: 13) | | |
| | subunit B(nheC) | NC-RI | TTT CCA GCT ATC TTT CGC TGT | | |
| | | | ATG TAA AT (SEQ ID NO: 14) | | |
| 3 | Enterotoxin FM | Fm-F1 | CAA AGA CTT CGT AAC AAA AGG | 160 | 290 |
| | (entFM) | | TGG T (SEQ D NO: 15) | | |
| | | Fm-R1 | TGT TTA CTC CGC CTT TTA CAA | | |
| | | | ACT T (SEQ ID NO: 16) | | |
| | Enterotoxin | Bcet-F1 | AGC TTG GAG CGG AGC AGA CTA | 700 | 701 |
| | T(bceT) | | TGT (SEQ ID NO: 17) | | |
| | | Bcet-RI | GTA TTT CTT TCC CGC TTG | | |
| | | | CCT TTT (SEQ ID NO: 18) | | |
| | Cytotoxln K(cytK) | Cyt-F1 | ATC GGG CAA AAT GCA AAA ACA | 500 | 800 |
| | | | CAT (SEQ ID NO: 19) | | |
| | | Cyt-R1 | ACC CAG TTT GCA GTT CCG AAT GT | | |
| | | | (SEQ ID NO: 20) | | |
| | Cytotoxln K(cytK) | Cyt-F2 | ATC GGT CAA AAT GCA AAA ACA | 500 | 800 |
| | | | CAT (SEQ ID NO: 21) | | |
| | | Cyt-R2 | ACC CAG TTA CCA GTT CCG AAT | | |
| | | | GT (SEQ ID NO: 22) | | |

TABLE 8
Results from Whole Genome Sequencing
Analysis of Bc and Bt Toxin Genes

| | GenBank | Query Result |
|---|---|---|
| Toxin Gene | Accesion # | for NRRL 68053 |
| hbl | U63928.1 | No significant |
| (subunits C/D) | | similarity found |
| nhe | DQ885236.1 | No significant |
| (subunits A/B/C) | | similarity found |
| cytK | AJ277962.1 | No significant |
| | | similarity found |
| entFM | AY789084.1 | No significant |
| | | similarity found |
| bceT | D17312.1 | No significant |
| | | similarity found |

### Example 12

### Cytoxicity Assays

EFSA requires Bacillus spp. be free of toxigenic activity. An in vitro cytotoxicity test was conducted on B. subtilis NRRL No. B-68053, using Vero (epithelial) cells. Cytotoxicity was measured with a lactate dehydrogenase (LDH) assay and was done in accordance with EFSA's “Guidance on the characterization of microogranisms used as feed additives or as production organisms”. Absorbance values above 20% of the absorbance obtained from the maximum LDH release control (Promega 10× Lysis Solution) indicates cytotoxicity. The supernatant from B. subtilis NRRL No. B-68053, at 10% concentration as shown in the tables below, had no cytotoxic effect against Vero cells.

TABLE 9
Bacillus subtilis NRRL No. B-68053 Percent LDH
Released by Vero Cells after 60 minutes exposure

| | Replicate | Replicate | Replicate | |
|---|---|---|---|---|
| Test Article | #1 | #2 | #3 | Average |
| B. subtilis NRRL No. | 5.52% | 1.98% | 2.02% | 3.18% |
| B-68053 % LDH | | | | |
| release | | | | |
| Untreated Control | 1.87% | 0.89% | 0.89% | 1.22% |

As a control for the endogenous LDH signal in the bacterial medium used to generate the test article, the test article was also tested in EC buffer (in the absence of Vero cells). The results of this assay are shown in the table above, as denoted in the equation below the table.

TABLE 10
Bacillus subtilis NRRL No. B-68053 Percent LDH
Signal in EC Buffer and Supernatant Controls

| | Replicate | Replicate | Replicate | |
|---|---|---|---|---|
| Test Article | #1 | #2 | #3 | Average |
| B. subtilis NRRL | 1.58% | 1.58% | 1.55% | 1.57% |
| No. B-68053 % | | | | |
| LDH release | | | | |

### Example 13

### RAPD-PCR DNA Profiles

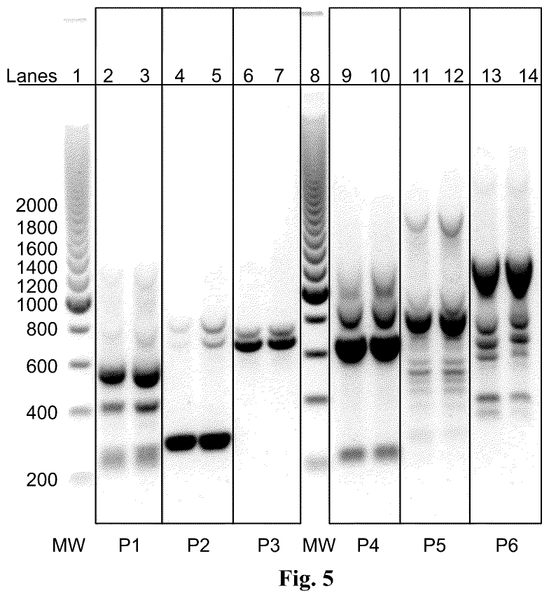

The randomly amplified polymorphic DNA PCR method (RAPD-PCR) was used to identify genetic variability of the strain. Preparation of the DNA to be used in the RAPD-PCR reaction was performed using Qiagen's Blood and Tissue single column kit. To obtain DNA, an overnight culture was prepared, struck for purity, pelleted, and the DNA was extracted following the manufacturer's protocol. Preparation of the RAPD-PCR reactions was done by using a Cytiva RAPD bead kit, which entails using one bead per reaction, sterile water, and a DNA template, along with one of six primers pre-designed to randomly amplify the polymorphic DNA. All six primers were used in separate reactions for each strain. Each 25 ul reactions contained 1 Cytiva RAPD bead, 16 ul sterile water, 5 ul of the respective primer, and 4 ul of the DNA template. Each sample was sealed, vortexed, and centrifuged briefly prior to running the reaction. The RAPD-PCR reaction was performed with the following run conditions in an AB2720 thermocycler; 95° C. 5 min, followed by 45 cycles of (95° C. 1 min, 36° C. 1 min, 72° C. 2 min), followed by 72° C. 7 min, and finished with a 4° C. indefinite hold to preserve the product. The RAPD-PCR product was analyzed by gel electrophoresis using 1% (wt/vol) agarose in 1×TBE buffer and a UV imager. The agarose gel contained SyberSafe Gel Stain at 1 ul/10 ml. The gel ran at 120 volts for 3 hours. At the end of the run, a digital camera attached to a UV imager uploaded the gel image to GeneSnap software which subsequently inverted the saturation values and adjusted the contrast for viewing purposes. See FIG. 5.

### Example 14

### Digestive Enyzme Production

Phenotypic enzyme assays using NRRL No. B-68053 resulted in positive enzymatic activity for lipase, amylase, protease, xylanase, and cellulase, and genomic analysis further yielded genomic potential for alpha and beta galactosidases, methionine reductases and synthases, uricase for uric acid, and prolyl endopeptidase for gluten degradation. These enzymes are extraordinary biocatalysts that increase the rate of biochemical reactions, particularly during digestion. The enzymes and reactions are responsible for hydrolyzing complex carbohydrates, lipids, and proteases down into more bioavailable components, thus increasing the rate of absorption and nutrient intake.

Phenotypic enzyme activity plate assays were performed to determine the enzymatic potential for digestive enzymes well-known to be produced by resident microbiota. Tryptic Soy Agar (TSA) was the base agar media used along with a selective agent specific to each of the enzymes. For lipase activity, 50 ml Tween 80 and 2.5 ml Polysorbate 80 were added to 1 L TSA. For amylase activity, 10 g of corn starch was added to 1 L TSA. For protease activity, 10 g of Casein was added to 1 L TSA. For xylanase activity, 10 g of Xylan was added to 1 L TSA. For cellulase activity, 5 g of CMC was added to 1 L TSA. All medium was autoclaved at 122° C. for 30 min and then poured and dried into agar plates. For each set of plates, 10 ul of NRRL No. B-68053 was spotted in triplicate, and incubated overnight at 37° C. The next day, positive enzymatic activity was determined by a zone of clearing around the culture spot. A negative reaction was indicated if there was no zone of clearing. Specific to xylanase activity, the culture plates were flooded with Gram's iodine, and positive enzymatic activity was determined by an unstained zone of clearing whereas a negative reaction was indicated by the entire plate become stained with the iodine.

### Example 15

### Antimicrobial Activity

NRRL No. B-68053 possesses antimicrobial activity against gram negative pathogens, gram positive pathogens, yeasts, and molds. Antimicrobial secondary metabolites are naturally produced and provide survival functions for organisms which produce them. Bacillus strains have been known to produce a wide diversity of antimicrobial secondary metabolites for decades, and are recognized as producing a spectra broader than those of lactic acid-producing bacteria. Antimicrobial susceptibility testing (AST) was performed using a cross-streak method. Bacillus strain NRRL No. B-68053 was inoculated from frozen glycerol stocks in a single 1 cm wide linear streak down the center of either TSA (E. coli, Salmonella, yeasts, and molds), or BHI+cysteine (Clostridium and Campylobacter) agar plates. Bacillus-streaked plates were incubated aerobically for 24 hrs at 37° C., until a heavy streak of growth was visible. The organisms to be tested for susceptibility were struck in lines perpendicular towards the Bacillus streak (up to 1 mm) in a biological safety cabinet and incubated by the following conditions per organism. Cross-streaks for the organisms E. coli and Salmonella were incubated aerobically at 37° C., yeast and mold strains were incubated aerobically at room temperature, Campylobacter strains were incubated microaerophillically at 42° C., and Clostridium and Enterococcus strains were incubated anaerobically at 37° C.; all for 24 hrs. No more than 5 staggered cross streaks per plate were applied. After the incubation period, plates were examined for zones of inhibition around the initial Bacillus streak, and the width of each zone of inhibition was quantitated in millimeters.

Bacillus subtilis strain NRRL No. B-68053 produced strong inhibition against Clostridium perfringens, Clostridium difficile, Candida albicans, and Candida auris strains. Medium inhibition was produced against E. coli, Salmonella, Enterococcus, Penicillium, and Aspergillus strains. Low inhibition was produced against Fusarium and Mucor strains.

### Example 16

### Immunomodulation

Bacillus species are known to modulate immunity through interactions with the host gastrointestinal tract. This immune modulation can help the human body keep a delicate balance between eliminating invading pathogens, while still maintaining a regulated level of inflammatory response capable of returning to homeostasis. Research has uncovered that the 70% of a human's immune system is localized within the epithelial tissues of the gastrointestinal tract. Bacillus, and particularly NRRL No. B-68053, are capable to communicating directly and indirectly with these immune cells in order to provide a necessary response to the surrounding environment, providing essential health benefits to the host, and regulating immune homeostasis.

The HT29 cell line was developed from human colorectal adenocarcinoma cells, and contrary to their Caco-2 epithelial cell counterpart, these cells secrete mucin which is important because the mucus layer has been suggested to play a role in modulating the adhesion of live organisms to the epithelial surface as well as bacterial cell components. HT29 cells represent a well characterized model to study the intestinal epithelial response to bacterial infection. This cell line expresses the features of enterocytes and mediates a response from many different immune cell types.

For testing the immunomodulation capacity of NRRL No. B-68053, the strain was exposed to HT29 cells in the presence and absence of LPS cell wall component. HT29 cells were cultured and passaged twice for consistency of viability using DMEM cell culture media supplemented with 10% FBS and 1% antibiotic/antimycotic and incubated at 37° C. with 5% CO₂. For the immune assay, cells were cultured in 24-wells plates, with 500 ul volumes, and seeded at a density of 100 k/well. A confluent monolayer was maintained for 21 days prior to exposure to allow for full maturation of the immune cells within the mucosal epithelial monolayer. Once maturity is reached, the antibiotic/antimycotic is removed from the media and given a four-hour equilibrium period.

To prep the NRRL No. B-68053 test article, an overnight culture was grown in 5 ml TSB and incubated at 37° C. The culture was quantitated using optical density measurements (OD at 600 nm), using an OD of 0.5 as the reference for 10⁷ cell counts for Bacillus. The sample was diluted using 1 mL sterile PBS to create 10⁶, 10⁵, 10⁴, and 10³ concentrations. Culture was also counted for exact cell counts. To prep the LPS compound, a thawed an aliquot of 100 ng/ul solution and diluted that down to 10 ng/ul using PBS. For the immune assay, 5 ul of 10 ng/ul LPS was used as the stimulation agent, and 10 ul of culture was used for each dose of the NRRL No. B-68053 strain. Each test condition and control had six replicates. Plates were incubated for 2 hrs at 37° C., 5% CO₂ (Table 11).

TABLE 11

| Plate #1 Setup | Plate #2 Setup |
|---|---|
| Control (No LPS, No NRRL | NRRL No. B-68053-dose 10⁴ |
| No. B-68053) | |
| Control + LPS (No NRRL | NRRL No. B-68053-dose 10⁴ + |
| No. B-68053) | LPS |
| NRRL No. B-68053-dose 10⁵ | NRRL No. B-68053-dose 10³ |
| NRRL No. B-68053-dose 10⁵ + | NRRL No. B-68053-dose 10³ + |
| LPS | LPS |

The cells were harvested after the two-hour incubation period, and RNA was extracted for expression analysis. Briefly, working with one well at a time as to go quickly and not allow the expression to change, media was removed and 200 ul of Tri reagent was added. The Tri reagent was allowed to work at room temperature for 5 minutes, then once the cells were released from being bound to the plate well, they were removed from the plate and placed into a 96-well 2 ml round bottom block. Block was covered with breathable sterile film at the first layer and an adhesive foil at the second layer. The block was then snap frozen in liquid nitrogen and stored at −80° C. until ready for RNA extraction and expression work.

RNA extraction was completed using Qiagen's RNeasy 96-well kit, using DTT added to the RLT buffer. RLT+DTT buffer was prepared by adding 40 ul/mL of 1M DTT into RLT buffer. The manufacturers protocol was followed, and two rounds of 45 ul was eluted. The RNA was stabilized into cDNA immediately after extraction using QuantaBio reverse transcriptase. For the reaction, 16 ul of RNA and 4 ul of rt-enzyme was added to a 96-well per plate. The reaction was ran on a Bio-Rad machine using a standard reverse transcription protocol provided by QuantaBio. For the immunomodulation assays, immune biomarker expression was quantitated and expressed as average relative quantity (RQ) values. GAPDH was used as a reference housekeeping gene. The results table below illustrates the average RQ value difference when compared to the LPS control.

TABLE 12
Results of Gene Expression Data for Immune Biomarkers

| Immune Biomarker | Biomarker Response | Average RQ Value e5 dose |
|---|---|---|
| TNFα | Pro-inflammatory | −33 |
| TLR4 | Pro-inflammatory | +0.5 |
| IL-6 | Pro-inflammatory | +2.12 |
| CXCL2 | Pro-inflammatory | −4 |
| SOCS1 | Anti-inflammatory | +.1 |
| TOLLIP | Anti-inflammatory | +1.51 |
| IL-10 | Anti-inflammatory | +1.79 |
| CXCL12 | Anti-inflammatory | +0.5 |

### Example 17

### Antioxidant Activity

Phenotypic antioxidant assays using NRRL No. B-68053 resulted in positive chelation and DPPH scavenging activity, indicating positive antioxidant capacity. Genomic analysis further yielded genomic potential for numerous natural enzymatic antioxidants, both primary (Superoxide dismutase genes against Copper, Iron, Zinc, and Manganese, Catalase activity, and Glutathione peroxidase) and Secondary (glucose-6-phosphate-dehydrogenase), and flavonoid activity (Quercetin).

While oxygen is a necessary element to many functions of living organisms, oxygen concentrations that exceed the normal range cause oxidative stress through the generation of reactive oxygen species (ROS). ROS is naturally produced through everyday occurrences both endogenously and exogenously. Endogenous sources include byproducts of metabolic processes, NADPH oxidases, mitochondrial electron transport chain leakage, and cytokine and growth factor receptors; while exogenous sources come from UV light, radiation, drugs, pollutants, and/or pathogens. The resulting oxidative stress can cause destructive damage to DNA/RNA, proteins, and lipids, and also result in cellular responses such as inflammation and carcinogenesis. If excessive amounts of ROS are not controlled, these destructive changes often lead to chronic diseases including atherosclerosis, arthritis, diabetes, Alzheimer's disease, neurodegenerative diseases, and cardiovascular diseases.

Antioxidants are ROS scavengers that can shield, scavenge, and repair oxidative damage, thereby defending target assemblies or molecules from oxidative damages. Living organisms have enzymatic and nonenzymatic antioxidant mechanisms for inactivating ROS, and microbes have been identified as a source for both. Enzymes, including catalase, glutathione peroxidase, and superoxide dismutase, are the endogenous antioxidants that control ROS damage, whereas carotenes, flavonoids, coenzyme Q, vitamins, minerals, and phenolic acids are the sources of exogenous antioxidants.

For testing antioxidant capacity of NRRL No. B-68053 phenotypically, we used assays quantifying DPPH scavenging activity and chelation activity. DPPH radical is a widely used method to evaluate the free radical scavenging ability of natural compounds. This assay is based on the measurement of the scavenging ability of antioxidant substances toward the stable radical. Ferrous ion chelation is another widely used assay to determine the scavenging potential.

For the DPPH assay, supernatants were prepared from NRRL No. B-68053 for use in these assays. This was completed by inoculation from 10 ul of an overnight culture grown in TSB, and then cultured for 24 hrs in a 250 mL flask with incubation conditions at a temperature of 32° C. and shaking at 180 rpm. The flasks were confirmed for purity, and centrifuged at 4° C., 6000 rpm for 20 minutes to separate the biomass from the supernatant. Supernatant was then filter sterilized through a 0.2 uM filter and aliquoted into 1.5 ml microcentrifuge tubes. The aliquots were snap frozen in liquid nitrogen and stored at −80° C. until use. Butylated Hydroxytoluene (BHT) was used as the reference standard and was prepared as a 1 mM solution in methanol. A standard curve was prepared from this stock solution in concentrations from 0-1000 uM in varying increments. 2,2-Diphenyl-1-picrylhydrazyl (DPPH) was prepared as a 0.1 mM working solution in methanol. For the assay, a 96-well microtiter was used, and 100 ul DPPH+ 100 ul of sample/standard was added to respective wells. The plate was wrapped in tin foil to protect from the light, and was incubated at room temperature of 60 minutes. Results were read using a plate reader at 517 nm wavelength. DPPH produces violet in methanol solution and fades to shades of yellow color in the presence of antioxidants. Percentage DPPH radical scavenging activity was calculated by the following equation below, whereas A₀ is the absorbance of the control, and A₁ is the absorbance of the extract/standard.

% DPPH radical scavenging activity={(A₀−A₁)/A₀}*100

Using the same supernatant extract prep as above, the Chelating Ferrous Ions assay was performed. EDTA was used as the reference standard was prepared as 1× working solution, from a 10× stock prepared by adding 0.05 g EDTA into 50 ml water, pH at 8.0 to dissolve. Additional reagents prepared were 10× stock Ferrozine (5 mM stock solution) and 10× stock FeCl₂ (2 mM stock solution). A n EDTA standard curve was prepared from the stock solution in concentrations of 0-50 mg/L in increments of 10 mg/L. For the experimental setup, a 96-well titer was used and contained a control, a control blank, a sample, and a sample blank. The control was comprised of 100 ul water+50 ul 1× FeCl₂+100 ul 1× Ferrozine. The control blank was comprised of 200 ul water+50 ul 1× FeCl₂. The Sample blank was comprised of 100 ul standard/sample+50 ul 1× FeCl₂+100 ul water. Finally, the sample was comprised of 100 ul standard/sample+50 ul 1× FeCl₂+100 ul 1× Ferrozine. All samples were run in triplicate. The plate was then incubated at room temperature for 5 minutes and read on a plate reader at 562 nm. The respective blanks were subtracted from the control and the sample, and percentage Chelating Activity was calculated by the following equation below, whereas A₀ is the absorbance of the control, and A₁ is the absorbance of the extract/standard. See Table 13.

% Chelating Activity={(A₀−A₁)/A₀}*100

TABLE 13
Results of Antioxidant Capacity Testing

| Phenotypic | % DPPH Activity | | 80.52% |
|---|---|---|---|
| Activity | % Chelating Activity | | 99.33% |
| Predicted | Primary | Enzymatic | SOD, CAT, GPx |
| Genomic | Antioxidants | | |
| Activity | Secondary | Enzymatic | Glucose-6-phosphate- |
| | Antioxidants | | dehydrogenase |

| | |
|---|---|
| Non-enzymatic Flavonoids | Quercetin |
| | 2,3-dioxygenase |

### Example 18

### Quorum Quenching Capacity

Quorum sensing is a cell's method of communicating, and is a cell density-dependent bacterial response mediated by autoinducer compounds. This communication network controls phenotypic variations including biofilm formation, virulence factor expression, and motility. Q uorum quenching is an organism's ability to inhibit or interfere with these communication using chemical or enzymatic means to counteract behaviors regulated by quorum sensing.

Both gram-negative and gram-positive bacterial organisms utilize this type of communication signaling, albeit through different peptide molecules. Gram-negative bacteria predominately utilize Acyl-hemoserine lactones (AHL) molecules such as AI-1, Luxl, or LuxR; while gram-positive bacteria predominately utilize autoinducing peptides (AIP). Chromobacterium violaceum is a well-studied quorum sensing reporter strain that harbors the LuxIR-type system to detect and respond to changes in cell population density. The hallmark trait of C. violaceum is its production of the purple pigment violacein, which is synthesized during active quorum sensing activity. However, when quorum sensing is inhibited, this reporter strain grows colorless, thus being a good screening tool for quorum quenching activity. In-vitro screening of NRRL No. B-68053 inhibition of C. violaceum quorum sensing communication was tested. Supernatants were prepared from NRRL No. B-68053 for use in these assays. This was completed by inoculation from 10 ul of an overnight culture grown in TSB, and then cultured for 24 hrs in a 250 ml flask with incubation conditions at a temperature of 32° C. and shaking at 180 rpm. The flasks were confirmed for purity, and centrifuged at 4° C., 6000 rpm for 20 minutes to separate the biomass from the supernatant. Supernatant was then filter sterilized through a 0.2 uM filter and aliquoted into 1.5 ml microcentrifuge tubes. The aliquots were snap frozen in liquid nitrogen and stored at −80° C. until use.

C. violaceum strain #12472 was purchased from ATCC and used as the reporter strain for this screening assay. Frozen stock culture was allowed to thaw at room temperature, in a biological safety cabinet (BSC). Additionally, frozen supernatant of NRRL No. B-68053 was allowed to thaw at room temperature, in a BSC. Using a 96-well microtiter plate, test wells were run as 180 ul of TSB, 20 ul NRRL No. B-68053 supernatant, and 2 ul ATCC 12472 C. violaceum strain. The positive control was 200 ul TSB and 2 ul C. violaceum. The negative control was 200 ul TSB. All samples were run in triplicate. The plate was incubated overnight, approximately 16 hrs, at 32° C. The next day, color differences were quantitated by reading the wells on a plate reader at an absorbance of 562 nm. Percentage quorum quenching activity was calculated by the following equation below, where A₀ is the absorbance of the control, and A₁ is the absorbance of the extract/standard. See Table 14.

% Quorum Quenching Activity={(A₀−A₁)/A₀}*100

TABLE 14
Results of Quorum Quenching Activity

| | | |
|---|---|---|
| Phenotypic Activity | % Quorum Quenching | 38.89% |
| | Activity | |

### Example 19

### Human Clinical Tolerability Study

A pilot, open-label design study assessed the safety and tolerability of Bacillus subtilis NRRL #68053 in healthy adult volunteers. See Tables 15 and 16. Ten participants were enrolled, and all ten participants completed the study. The participants had an average age of 35.1±11.6 years, weight of 75.6±7.1 kilograms, and body mass index of 23.8±1.5. The basic inclusion criteria stated participants must be in good health as determined by medical history and routine blood chemistries, and to maintain their regular diet and exercise patterns for the duration of the study. The study also included more defined inclusion/exclusion criteria around medical history, medications, and over the counter supplements. The compliance and completion rate of participants was 100%.

Participants were given daily capsules of 1 Billion total CFU to be taken for 42 days. The study product used maltodextrin as the carrier and size 0 vegetarian capsules. Participants were instructed to take the capsules on a 24-hour cadence, preferably with a meal. The primary endpoints were monitoring for adverse events and weekly visual analogue scale (VAS) questionnaires to assess GI health and comfort, mood, and stress scores. GI health questionnaires recorded scores for flatulence, bloating, abdominal discomfort, stool consistency, stool regularity, bowel frequency, and constipation. Mood and Stress questionnaires recorded scores for enthusiasm, well-being, vigor, fatigue, depression, anxiety, restlessness, nervousness, worrying, and feelings of doom. Additional secondary and tertiary endpoints measured various blood biomarkers to assess trends.

Adverse Events were recorded on a weekly basis throughout the study and assessed on a scale of mild to severe (1-5), along with CBC blood and lipid panels, and vital signs for overall health. No severe adverse events were reported during the study. One mild case of constipation was reported as possibly related to test article. The consumption of Bacillus subtilis NRRL #68053 at a dose of 1×10⁹ CFU per day for 42 days to healthy adult volunteers was concluded to be well tolerated.

Study results were evaluated through statistical analysis performed using JASP version 0.17.2.1. Statistical significance was determined by a one-way ANOVA, Tukey comparison with 95% confidence intervals. For the gut health questionnaires, Bacillus subtilis NRRL #68053 significantly improved scores for flatulence and bloating, and further yielded trending improvements for abdominal discomfort and directional improvements for stool regulatory. There were no differences in constipation levels recorded. For mood and stress questionnaires, Bacillus subtilis NRRL #68053 significantly improved levels of enthusiasm, and significantly decreased feelings of fatigue and anxiety. Furthermore, trending improvements in the decrease of worrying and irritability, and directional improvements for increasing feelings of well-being and vigor; and decreasing feelings of depression, nervousness, and trouble relaxing were recorded over the six-week span. There were no differences in levels of restlessness.

Additional blood serum was collected and analyzed for immunomodulatory biomarkers, and as expected there was a substantial amount of biological variability from person to person, especially in the cytokine markers IL-2, IL-8, and IL-10. However, there was a significant decrease in the inflammatory cytokine TNFα between the initial testing and final testing after the six-week study duration.

TABLE 15
Summary of outcome means, standard deviations, and statisical significance.

| N = 10 (males, | Gut Health | | | Mood & Stress | | | Immune Biomarkers | | |
|---|---|---|---|---|---|---|---|---|---|
| 22-50 yo) | Outcome | Day 0 | Day 42 | Outcome | Day 0 | Day 42 | Outcome | Day 0 | Day 42 |
| Significant | Flatulence | 4.29 | 2.59 | Enthusiasm | 51.3 | 7.11 | TNFα | 8.41 | 4.47 |
| Improvements | | (±1.6) | (±1.8) | | (±2.2) | (±1.7 | | (±0.8) | (±1.5) |
| (p value ≤ | Bloating | 3.98 | 2.00 | Fatigue | 5.17 | 3.12 | | | |
| 0.05) | | (±2.1) | (±1.9) | | (±2.7) | (±1.4) | | | |
| | | | | Anxiety | 4.14 | 19.6 | | | |
| | | | | | (±2.7) | (±1.4 | | | |
| Trending | Abdominal | 4.19 | 2.35 | Worrying | 1.30 | 0.50 | | | |
| Improvements | discomfort | (±2.4) | (±2.0) | | (±1.2) | (±0.9) | | | |
| (0.05 < p | | | | Irritability | 0.90 | 0.30 | | | |
| value ≤ 0.1) | | | | | (±0.9) | (±0.4) | | | |
| | | | | GAD-7 Score | 7.1 | 3.00 | | | |
| | | | | | (±6.4) | (±2.2) | | | |
| Directional | Stool | 4.49 | 6.25 | Well-being | 6.10 | 7.42 | | | |
| Improvements | Regularity | (±2.6) | (±2.4) | | (±2.1) | (±1.6) | | | |
| (0.1 < p | | | | Vigor | 5.90 | 3.10 | | | |
| value ≤ 0.2) | | | | | (±2.4) | (±2.5) | | | |
| | | | | Trouble | 1.20 | 0.70 | | | |
| | | | | Relaxing | (±0.9) | (±0.5) | | | |
| | | | | Nervousness | 1.00 | 0.60 | | | |
| | | | | | (±0.8) | (±0.5) | | | |
| | | | | Depression | 3.10 | 1.53 | | | |
| | | | | | (±2.5) | (±1.7) | | | |
| No Differences | Constipation | 2.23 | 2.14 | Restlessness | 0.90 | 0.50 | IL2 | 297 | 276 |
| (p value ≥ 0.2) | | (±2.2) | (±1.5) | | (±1.2) | (±0.5) | | (±169) | (±147) |
| | | | | | | | IL6 | 1.03 | 0.69 |
| | | | | | | | | (±0.9) | (±0.8) |
| | | | | | | | IL8 | 40.2 | 29.4 |
| | | | | | | | | (±47.5) | (±43.2) |
| | | | | | | | IFNy | 4.27 | 3.33 |
| | | | | | | | | (±3.4) | (±3.5) |
| | | | | | | | IL10 | 14.7 | 14.9 |
| | | | | | | | | (±41.9) | (±42.9) |

TABLE 16
Illustration of outcome means; when larger
is best, and when smaller is best.

| | Turkey Comparison: Hsu MCB | |
|---|---|---|
| Outcomes: | Largest Mean is best | Smallest mean is best |
| Gut Health | | |
| Stool Regularity | X | |
| Flatulence | | X |
| Bloating | | X |
| Abdominal Discomfort | | X |
| Constipation | | X |
| Mood & Stress | | |
| Enthusiasm | X | |
| Well-being | X | |
| Vigor | X | |
| Fatigue | | X |
| Depression | | X |
| Anxiety | | X |
| Nervousness | | X |
| Worrying | | X |
| Trouble Relaxing | | X |
| Restlessness | | X |
| Irritability | | X |
| GAD-7 Total Score | | X |
| Immune Biomarkers | | |
| IL-10 | X | |
| IL-2 | | X |
| IL-6 | | X |
| IL-8 | | X |
| INFa | | X |
| IFNy | | X |

Bacillus Strain (NRRL No. B-68054)

### Example 20

### Antibiotic Resistance

The inventors have assessed resistance phenotypically. A bacterial strain is defined by EFSA as susceptible when its growth is inhibited at concentration of a specific antibiotic that is equal to or lower than the established cut-off value for that particular species. A bacterial strain is defined as resistant when it is able to grow at a concentration of a specific antibiotic that is higher than the established cut-off. The inventors utilized both the EFSA and CLSI guidelines for determining susceptibility and resistance interpreted through their defined cut-off values.

TABLE 17
Results of Minimum Inhibitory Concentrations (MICs) Against Medical Antibiotics

| | NRRL | | | | |
|---|---|---|---|---|---|
| | No. | EFSA | NRRL No. | CLSI | NRRL No. |
| | B-68054 | Breakpoints | B-68054 | Breakpoints for | B-68054 |
| | ug/mL | for Bacillus | EFSA | Staphylococcus | CLSI |
| Antibiotic | (triplicate) | spp. | Interpretation | spp. | Interpretation |
| Chloramphenicol | 1.5 | 8 | Sensitive | ≤8 | Sensitive |
| Clindamycin | 0.5 | 4 | Sensitive | ≤0.5 | Sensitive |
| Ciprofloxacin | 0.064 | n.r. | n.r. | ≤1 | Sensitive |
| Erythromycin | 0.047 | 4 | Sensitive | ≤0.5 | Sensitive |
| Gentamicin | 0.125 | 4 | Sensitive | ≤4 | Sensitive |
| Kanamycin | 0.75 | 8 | Sensitive | ≤16 | Sensitive |
| Linezolid | 0.25 | n.r. | n.r. | ≤4 | Sensitive |
| Rifampin | 0.5 | n.r. | n.r. | ≤1 | Sensitive |
| Streptomycin | 4 | 8 | Sensitive | n.r. | n.r. |
| Tetracycline | 6 | 8 | Sensitive | ≤4 | Intermediate |
| Tigecycline | 0.064 | n.r. | n.r. | ≤0.5 | Sensitive |
| Vancomycin | 0.75 | 4 | Sensitive | ≤2 | Sensitive |

### Example 21

### Hemolysis Activity

Bacillus strain (NRRL No. B-68054) was tested for hemolysis activity by methods of blood agar plates to determine zero, partial, or complete lysis of the red blood cells. NRRL 68054 was negative for hemolysis activity. Beta-hemolysis is defined as complete or true lysis of red blood cells. A clear zone, approaching the color and transparency of the base medium, surrounds the colony. Alpha-hemolysis is the reduction of the red blood cell hemoglobin to methemoglobin the medium surrounding the colony. Discoloration resulting in brown or green colored cells surrounding the medium is common of alpha hemolysis. Unlike beta hemolysis, alpha hemolysis maintains the structure of the cell membrane.

NRRL No. B-68054 was cultured and tested for antibiotic susceptibility by the following methods. First, the strain was cultured from a frozen stock by using a sterile inoculating loop to iso-streak the cells on tryptic soy agar media and incubated overnight at 37° C. When the colonies were visible the next day, they were morphologically examined for purity and a sterile inoculating loop was used to collect a single colony. The single colony was cultured to a higher density by inoculating it into tryptic soy broth and incubated overnight at 37° C., 230 rpm. The next day, the growth culture was standardized to a cell density of OD_{600 nm} 0.8 using 0.1% peptone as a diluent. The blood agar plates were purchased pre-made and comprised of tryptic soy agar with 5% sheep's blood. Using a sterile inoculating loop with a diameter that holds approximately 10 μl, the culture was streaked horizontally across the surface of plate, in triplicate. The plates were then incubated overnight at 37° C. The next day, the plates were removed from the incubator and results were recorded. Results were determined as ‘no hemolysis’ for no discoloration of the media, ‘alpha hemolysis’ partial hemolysis and green-brown discoloration of the media, and ‘beta hemolysis’ for complete lysis of the media and display of clearing zone around the cells. NRRL No. B-68054 produced no hemolysis around the cells streaked on the blood agar plate for all three triplicate streaks.

### Example 22

### Enterotoxins

Bacillus strain (NRRL No. B-68054) resulted in negative presence for all known toxin genes commonly associated with Bacillus cereus and Bacillus thuringiensis using a Multiplex PCR method adapted from Yang et al. Whole genome sequence analysis was also used to query for these known Bacillus toxin gene sequences and the database yielded no significant similarities found. NRRL No. B-68054 was cultured, DNA was extracted, and tested for toxins by the following methods. First, the strain was cultured from a frozen stock by using a sterile inoculating loop to iso-streak the cells on tryptic soy agar media and incubated overnight at 37° C. When the colonies were visible the next day, they were morphologically examined for purity and a sterile inoculating loop was used to collect a single colony to extract DNA from the strain. The single colony was cultured to a higher density by inoculating it into tryptic soy broth and incubated overnight at 37° C. The next day, 1 ml of the culture was transferred to a 1.5 ml microcentrifuge tube, and the cells were pelleted in a centrifuge at 10,000 rpm for 10 minutes. DNA extraction of those cells were carried using Qiagen's DNeasy Blood and Tissue single column kit. The supernatant from the pelleted cells was discarded and the pellet was resuspended in 180 μl of lysis buffer containing 20 mg/ml of lysozyme. NRRL No. B-68054+lysis solution was incubated at 37° C. for 45 minutes to break apart the cell walls and lyse the cellular components. Then, 20 μl proteinase K and 200 μl Buffer AL were added and sample was incubated for an additional 30 minutes at 56° C. to degrade proteinaceous components. Upon completion of incubation, 200 μl EtOH was added and vortexed to a homogenous solution to aggregate insoluble DNA. Spin columns were used at the manufacturer's recommended protocol to separate and clean DNA from the solution. The DNA was eluted in 100 μl Low-TE Buffer purchased from ThermoFisher.

The primer sequences and multiplex PCR methods were adapted from Yang et al. Primers for toxins Hemolysin BL subunit A (hb1A), Hemolysin BL subunit C (hb1C.), Hemolysin BL subunit D (hb1D), Nonhemolytic enterotoxin subunit A (nheA), Nonhemolytic enterotoxin subunit B (nheB), Nonhemolytic enterotoxin subunit C (nheC), Emetic toxin, Enterotoxin FM (entFM), Enterotoxin T (bceT), and Cytotoxin K (cytK) to test presence of Bacillus spp. producing toxins. These primer sequences were purchased from Eurofins and diluted to working concentrations 40-350 nM (Table 18). Three separate master mixes were prepared for the multiplex toxin assays as seen in Table 18. For each master mix, the total volume was 49 μl, comprising of 200 μM DNTPs, 1×PCR Buffer, 2U fast start Taq polymerase, and the remaining volume with ddH₂O. The total reaction volume was 55 μl, comprising of 49 μl of the respective master mix and 6 μl of template DNA. Bacillus cereus and Bacillus thuringiensis genomic DNA were used as positive controls, and ddH₂O was used for no template controls.

The PCR reaction was performed on an Applied Biosystems 2720 Thermal Cycler utilizing endpoint PCR technologies. All three master mixes were tested against the NRRL No. B-68054, Bc positive control, Bt positive control, and NT negative control. The reaction conditions consisted of a denaturation step at 95° C. for 5 min followed by 30 cycles of 95° C. for 30 seconds, 60° C. for 30 seconds, 72° C. for 45 seconds, and finished with an elongation step at 72° C. for 7 minutes. The amplification products were analyzed on a 2% agarose gel, with electrophoresis for 80 minutes at 100 volts, and visualized using a Gel Imaging System with UV transilluminator and GeneSnap software tools.

NRRL No. B-68054 genomic DNA was negative for presence of any toxins within the three multiplex assays through 30 cycles of amplification, shown in FIG. 7. The genomic DNA controls for Bc and Bt yielded amplification products at the anticipated band sizes for each of the of the multiplexed assays, and the no template control was negative in all three master mixes. Results of whole genome sequence analysis yielded no significant similarity of NRRL No. B-68054 with gene sequences for these known Bacillus cereus and Bacillus thuringiensis toxins (Table 19).

The multiplex PCR assay yielded no amplification for Bacillus cereus-like toxin genes Hemolysin BL, Nonhemolytic Enterotoxin, Enterotoxin FM, Enterotoxin T, nor Cytotoxin K for NRRL No. B-68054 DNA. Amplified PCR products were seen at expected band sizes and additionally there were no stray bands at unexpected sizes for the genomic DNA positive controls, all with no amplification was seen in the no template control. Furthermore, as stated in Yang et al., the sensitivity of the multiplex PCR assay was approximately 100 pg and the primers were specific to the designed target. All of this data suggests that these primer sequences, paired in multiplex reactions, are an adequate method of analysis. These results were confirmed by blasting the whole genome sequence against the toxin gene sequencing via BLASTn database, displayed in Table 18.

TABLE 18
Master Mix Groups and Primer Concentrations for Each Primer Set

| | | Primer | | Concentration | Fragments |
|---|---|---|---|---|---|
| Set | Targeted gene | name | Sequence (5′ to 3′) | used (nM) | size (BP) |
| 1 | Hemolysin BL | HA-F1 | ATT AAT ACA GGG GAT GGA GAA | 150 | 237 |
| | subunit A | | ACT T (SEQ ID NO: 1) | | |
| | (hbLA) | HA-RI | TGA TCC TAA TAC TTC TTC TAG | | |
| | | | ACG CTT (SEQ ID NO: 2) | | |
| | Hemolysin BL | HC-F1 | CCT ATC AAT ACT CTC GCA ACA | 240 | 386 |
| | subunit C | | CCA AT (SEQ ID NO: 3) | | |
| | (hbLC) | HC-RI | TTT TCT TGA TTC GTC ATA GCC | | |
| | | | ATT TCT (SEQ ID NO: 4) | | |
| | Hemolysin BL | HD-F1 | AGA TGC TAC AAG ACT TCA AAG | 240 | 436 |
| | subunit D | | GGA AAC TAT (SEQ ID NO: 5) | | |
| | (hbLD) | HD-A1 | TGA TTA GCA CGA TCT GCT TTC | | |
| | | | ATA CTT (SEQ ID NO: 6) | | |
| | Emetic strain- | EM-F1 | AGC TTG GAG CGG AGC AGA CTA | 500 | 635 |
| | specific | | TGT (SEQ ID NO; 7) | | |
| | sequence | EM-RI | GTA TTT CTT TCC CGC TTG CCT TTT | | |
| | | | (SEQ ID NO: 8) | | |
| 2 | Nonhemolytlc | NA-FI | ATT ACA GGG TTA TTG GTT ACA | 150 | 475 |
| | enterotoxin | | GCA GT (SEQ ID NO: 9) | | |
| | subunit A(nheA) | NA-RI | AAT CTT GCT CCA TAC TCT CTT | | |
| | | | GGA TGC T (SEQ ID NO: 10) | | |
| | Nonhemolytlc | NB-F1 | GTG CAG CAG CTG TAG GCG GT | 180 | 328 |
| | enterotoxin | | (SEQ ID NO: 11) | | |
| | subunit B(nheB) | NB-R1 | ATG TTT TTC CAG CTA TCT TTC | | |
| | | | GCA AT (SEQ ID NO: 12) | | |
| | Nonhemolytlc | NC-F1 | GCG GAT ATT GTA AAG AAT CAA | 450 | 557 |
| | enterotoxin | | AATGAG GT (SEQ ID NO: 13) | | |
| | subunit B(nheC) | NC-RI | TTT CCA GCT ATC TTT CGC TGT | | |
| | | | ATG TAA AT (SEQ ID NO: 14) | | |
| 3 | Enterotoxin FM | Fm-F1 | CAA AGA CTT CGT AAC AAA AGG | 160 | 290 |
| | | | TGG T (SEQ D NO: 15) | | |
| | (entFM) | Fm-R1 | TGT TTA CTC CGC CTT TTA CAA | | |
| | | | ACT T (SEQ ID NO: 16) | | |
| | Enterotoxin T(bceT) | Bcet-F1 | AGC TTG GAG CGG AGC AGA CTA | 700 | 701 |
| | | | TGT (SEQ ID NO: 17) | | |
| | | Bcet-RI | GTA TTT CTT TCC CGC TTG | | |
| | | | CCT TTT (SEQ ID NO: 18) | | |
| | Cytotoxln K (cytK) | Cyt-F1 | ATC GGG CAA AAT GCA AAA ACA | 500 | 800 |
| | | | CAT (SEQ ID NO: 19) | | |
| | | Cyt-R1 | ACC CAG TTT GCA GTT CCG AAT GT | | |
| | | | (SEQ ID NO: 20) | | |
| | Cytotoxln K (cytK) | Cyt-F2 | ATC GGT CAA AAT GCA AAA ACA | 500 | 800 |
| | | | CAT (SEQ ID NO: 21) | | |
| | | Cyt-R2 | ACC CAG TTA CCA GTT CCG AAT | | |
| | | | GT (SEQ ID NO: 22) | | |

TABLE 19
Results from Whole Genome Sequencing
Analysis of Bc and Bt Toxin Genes

| | GenBank | Query Result for NRRL |
|---|---|---|
| Toxin Gene | Accesion # | No. B-68054 |
| hbl (subunits C/D) | U63928.1 | No significant similarity |
| | | found |
| nhe (subunits A/B/C) | DQ885236.1 | No significant similarity |
| | | found |
| cytK | AJ277962.1 | No significant similarity |
| | | found |
| entFM | AY789084.1 | No significant similarity |
| | | found |
| bceT | D17312.1 | No significant similarity |
| | | found |

### Example 23

### Cytoxicity Assays

EFSA requires Bacillus spp. be free of toxigenic activity. An in vitro cytotoxicity test on B. subtilis NRRL No. B-68054 was conducted, using Vero (epithelial) cells. Cytotoxicity was measured with a lactate dehydrogenase (LDH) assay and was done in accordance with EFSA's “Guidance on the characterization of microogranisms used as feed additives or as production organisms”. Absorbance values above 20% of the absorbance obtained from the maximum LDH release control (Promega 10× Lysis Solution) indicates cytotoxicity. The supernatant from B. subtilis NRRL No. B-68054, at 10% concentration as shown in the tables below, had no cytotoxic effect against Vero cells.

TABLE 20
Bacillus subtilis NRRL No. B-68054 Percent LDH
Released by Vero Cells after 60 minutes exposure

| | Replicate | Replicate | Replicate | |
|---|---|---|---|---|
| Test Article | #1 | #2 | #3 | Average |
| B. subtilis NRRL No. | 2.75% | 2.31% | 2.71% | 2.59% |
| B-68054 % LDH | | | | |
| release | | | | |
| Untreated Control | 1.87% | 0.89% | 0.89% | 1.22% |

As a control for endogenous LDH signal in the bacterial medium used to generate the test article, the test article was also tested in EC buffer (in the absence of Vero cells). The results of this assay are shown in the table above, as denoted in the equation below the table.

TABLE 21
Bacillus subtilis NRRL No. B-68054 Percent LDH
Signal in EC Buffer and Supernatant Controls

| | Replicate | Replicate | Replicate | |
|---|---|---|---|---|
| Test Article | #1 | #2 | #3 | Average |
| B. subtilis NRRL | 1.93% | 1.91% | 2.09% | 1.98% |
| No. B-68054 % | | | | |
| LDH release | | | | |

### Example 24

### RAPD-PCR DNA Profiles

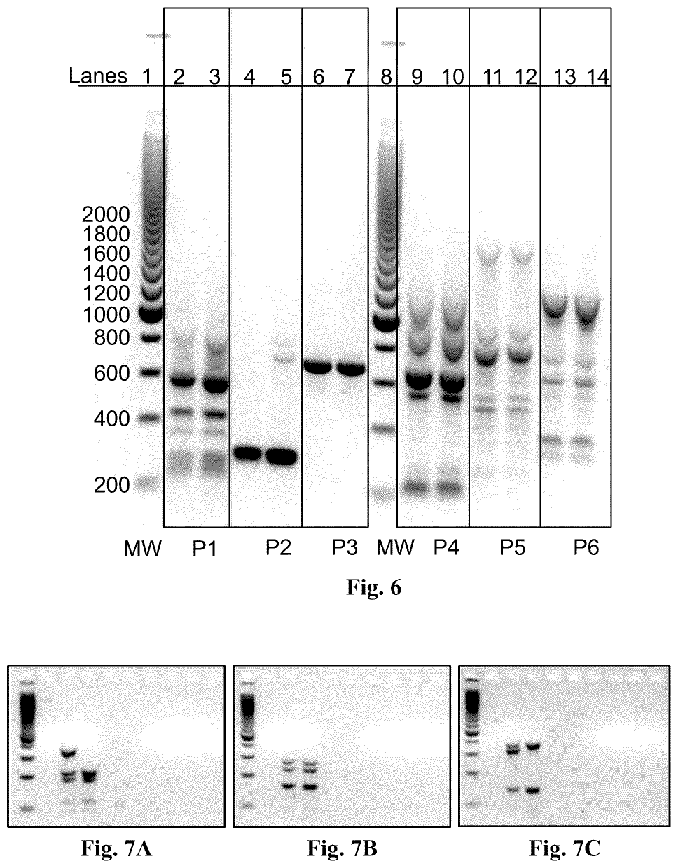

The randomly amplified polymorphic DNA PCR method (RAPD-PCR) was used to identify genetic variability of the strain NRRL No. B-68054. Preparation of the DNA to be used in the RAPD-PCR reaction was performed using Qiagen's Blood and Tissue single column kit. To obtain DNA, an overnight culture was prepared, struck for purity, pelleted, and DNA was extracted following the manufacturer's protocol. Preparation of the RAPD-PCR reactions was done by using a Cytiva RAPD bead kit, which entails using one bead per reaction, sterile water, and the DNA template, along with one of six primers pre-designed to randomly amplify the polymorphic DNA. All six primers were used in separate reactions for each strain. Each 25 ul reactions contained 1 Cytiva RAPD bead, 16 ul sterile water, 5 ul respective primer, and 4 ul of the DNA template. Each sample was sealed, vortexed, and centrifuged briefly prior to running the reaction. The RAPD-PCR reaction was performed with the following run conditions in an AB2720 thermocycler; 95° C. 5 min, followed by 45 cycles of (95° C. 1 min, 36° C. 1 min, 72° C. 2 min), followed by 72° C. 7 min, and finished with a 4° C. indefinite hold to preserve the product. The RAPD-PCR product was analyzed by gel electrophoresis using 1% (wt/vol) agarose in 1×TBE buffer and a UV imager. The agarose gel contained SyberSafe Gel Stain at 1 ul/10 ml. The gel ran at 120 volts for 3 hours. At the end of the run, a digital camera attached to a UV imager uploaded the gel image to GeneSnap software which subsequently inverted the saturation values and adjusted the contrast for viewing purposes. See FIG. 6.

### Example 25

### Digestive Enyzme Production

Phenotypic enzyme assays using NRRL No. B-68054 resulted in positive enzymatic activity for Lipase, Amylase, Protease, Xylanase, and Cellulase, and genomic analysis further yielded genomic potential for alpha and beta galactosidases, methionine reductases and synthases, uricase for uric acid, and prolyl endopeptidase for gluten degradation. These enzymes are extraordinary biocatalysts that increase the rate of biochemical reactions, particularly during digestion. The enzymes and reactions are responsible for hydrolyzing complex carbohydrates, lipids, and proteases down into more bioavailable components, thus increasing the rate of absorption and nutrient intake.

Phenotypic enzyme activity plate assays were performed to determine the enzymatic potential for digestive enzymes well-known to be produced by resident microbiota. Tryptic Soy Agar (TSA) was the base agar media used along with a selective agent specific to each of the enzymes. For lipase activity, 50 ml Tween 80 and 2.5 ml Polysorbate 80 were added to 1 L TSA. For amylase activity, 10 g of corn starch was added to 1 L TSA. For protease activity, 10 g of casein was added to 1 L TSA. For xylanase activity, 10 g of Xylan was added to 1 L TSA. For cellulase activity, 5 g of CMC was added to 1 L TSA. All medium was autoclaved at 122° C. for 30 min and then poured and dried into agar plates. For each set of plates, 10 ul of NRRL 68054 was spotted in triplicate, and incubated overnight at 37° C. The next day, positive enzymatic activity was determined by a zone of clearing around the culture spot. A negative reaction was indicated if there was no zone of clearing. Specific to xylanase activity, the culture plates were flooded with Gram's iodine, and positive enzymatic activity was determined by an unstained zone of clearing whereas a negative reaction was indicated by the entire plate become stained with the iodine.

### Example 26

### Immunomodulation

Bacillus species are known to modulate immunity through interactions with the host gastrointestinal tract. This immune modulation can help the human body keep a delicate balance between eliminating invading pathogens, while still maintaining a regulated level of inflammatory response capable of returning to homeostasis. Research has uncovered that the 70% of a human's immune system is localized within the epithelial tissues of the gastrointestinal tract. Bacillus, and particularly NRRL No. B-68054, are capable to communicating directly and indirectly with these immune cells in order to provide a necessary response to the surrounding environment, providing essential health benefits to the host, and regulating immune homeostasis.

The HT29 cell line was developed from human colorectal adenocarcinoma cells, and contrary to their Caco-2 epithelial cell counterpart, these cells secrete mucin which is important because the mucus layer has been suggested to play a role in modulating the adhesion of live organisms to the epithelial surface as well as bacterial cell components. HT29 cells represent a well characterized model to study the intestinal epithelial response to bacterial infection. This cell line expresses the features of enterocytes and mediates a response from many different immune cell types.

For testing the immunomodulation capacity of NRRL No. B-68054, the strain was exposed to HT29 cells in the presence and absence of LPS cell wall component. HT29 cells were cultured and passaged twice for consistency of viability using DMEM cell culture media supplemented with 10% FBS and 1% antibiotic/antimycotic and incubated at 37° C. with 5% CO₂. For the immune assay, cells were cultured in 24-wells plates, with 500 ul volumes, and seeded at a density of 100 k/well. A confluent monolayer was maintained for 21 days prior to exposure to allow for full maturation of the immune cells within the mucosal epithelial monolayer. Once maturity is reached, the antibiotic/antimycotic is removed from the media and given a four-hour equilibrium period.

To prep the NRRL No. B-68054 test article, an overnight culture was grown in 5 ml TSB and incubated at 37° C. The culture was quantitated using optical density measurements (OD at 600 nm), using an OD of 0.5 as the reference for 10⁷ cell counts for Bacillus. The sample was diluted using 1 mL sterile PBS to create 10⁶, 10⁵, 10⁴, and 10³ concentrations. Culture was also counted for exact cell counts. To prep the LPS compound, a thawed an aliquot of 100 ng/ul solution and diluted that down to 10 ng/ul using PBS. For the immune assay, 5 ul of 10 ng/ul LPS was used as the stimulation agent, and 10 ul of culture was used for each dose of the NRRL No. B-68054 strain, as shown below. Each test condition and control had six replicates. Plates were incubated for 2 hrs at 37° C., 5% CO₂.

| Plate #1 Setup | Plate #2 Setup |
|---|---|
| Control (No LPS, No NRRL | NRRL No. B-68054-dose 10⁴ |
| No. B-68054) | |
| Control + LPS (No NRRL | NRRL No. B-68054-dose 10⁴ + |
| No. B-68054) | LPS |
| NRRL No. B-68054-dose 10⁵ | NRRL No. B-68054-dose 10³ |
| NRRL No. B-68054-dose 10⁵ + | NRRL No. B-68054-dose 10³ + |
| LPS | LPS |

The cells were harvested after the two-hour incubation period, and RNA was extracted for expression analysis. Briefly, working with one well at a time as to go quickly and not allow the expression to change, media was removed and 200 ul of Tri reagent was added. The Tri reagent was allowed to work at room temperature for 5 minutes, then once the cells were released from being bound to the plate well, they were removed from the plate and placed into a 96-well 2 ml round bottom block. The block was covered with breathable sterile film at the first layer and an adhesive foil at the second layer. The block was then snap frozen in liquid nitrogen and stored at −80° C. until ready for RNA extraction and expression work.

RNA extraction was completed using Qiagen's RNeasy 96-well kit, using DTT added to the RLT buffer. RLT+DTT buffer was prepared by adding 40 ul/ml of 1M DTT into RLT buffer. The manufacturers protocol was followed, and two rounds of 45 ul was eluted. The RNA was stabilized into cDNA immediately after extraction using QuantaBio reverse transcriptase. For the reaction, 16 ul of RNA and 4 ul of rt-enzyme was added to a 96-well per plate. The reaction was ran on a Bio-Rad machine using a standard reverse transcription protocol provided by QuantaBio. For the immunomodulation assays, immune biomarker expression was quantitated and expressed as average relative quantity (RQ) values. GAPDH was used as a reference housekeeping gene. The results table below (Table 22) illustrates the average RQ value difference when compared to the LPS control.

TABLE 22
Results of Gene Expression Data for Immune Biomarkers

| Immune Biomarker | Biomarker Response | Average RQ Value e5 dose |
|---|---|---|
| TNFa | Pro-inflammatory | −38 |
| TLR4 | Pro-inflammatory | −0.3 |
| IL-6 | Pro-inflammatory | +3.06 |
| CXCL2 | Pro-inflammatory | −5 |
| SOCS1 | Anti-inflammatory | +.41 |
| TOLLIP | Anti-inflammatory | +2.2 |
| IL-10 | Anti-inflammatory | +1.3 |
| CXCL12 | Anti-inflammatory | +1.2 |

### Example 27

### Antioxidant Activity

Phenotypic antioxidant assays using NRRL No. B-68054 resulted in positive chelation and DPPH scavenging activity, indicating positive antioxidant capacity. Genomic analysis further yielded genomic potential for numerous natural enzymatic antioxidants, both primary (Superoxide dismutase genes against Copper, Iron, Zinc, and Manganese, Catalase activity, and Glutathione peroxidase) and Secondary (glucose-6-phosphate-dehydrogenase), and flavonoid activity (Quercetin).

While oxygen is a necessary element to many functions of living organisms, oxygen concentrations that exceed the normal range causes oxidative stress through the generation of reactive oxygen species (ROS). ROS is naturally produced through everyday occurrences both endogenously and exogenously. Endogenous sources include byproducts of metabolic processes, NADPH oxidases, mitochondrial electron transport chain leakage, and cytokine and growth factor receptors; while exogenous sources come from UV light, radiation, drugs, pollutants, and/or pathogens. The resulting oxidative stress can cause destructive damage on DNA/RNA, proteins, and lipids, and also result in cellular responses such as inflammation and carcinogenesis. If excessive amounts of ROS are not controlled, these destructive changes often lead to chronic diseases including atherosclerosis, arthritis, diabetes, Alzheimer's disease, neurodegenerative diseases, and cardiovascular diseases.

Antioxidants are ROS scavengers that can shield, scavenge, and repair oxidative damage, thereby defending target assemblies or molecules from oxidative damages. Living organisms have enzymatic and nonenzymatic antioxidant mechanisms for inactivating ROS, and microbes have been identified as a source for both. Enzymes, including catalase, glutathione peroxidase, and superoxide dismutase, are the endogenous antioxidants that control ROS damage, whereas carotenes, flavonoids, coenzyme Q, vitamins, minerals, and phenolic acids are the sources of exogenous antioxidants.

For testing antioxidant capacity of NRRL No. B-68054 phenotypically, we used assays quantifying DPPH scavenging activity and chelation activity. DPPH radical is a widely used method to evaluate the free radical scavenging ability of natural compounds. This assay is based on the measurement of the scavenging ability of antioxidant substances toward the stable radical. Ferrous ion chelation is another widely used assay to determine the scavenging potential.

For the DPPH assay, supernatants were prepared from NRRL No. B-68054 for use in these assays. This was completed by inoculation from 10 ul of an overnight culture grown in TSB, and then cultured for 24 hrs in a 25 ml flask with incubation conditions at a temperature of 32° C. and shaking at 180 rpm. The flasks were confirmed for purity, and centrifuged at 4° C., 6000 rpm for 20 minutes to separate the biomass from the supernatant. Supernatant was then filter sterilized through a 0.2 uM filter and aliquoted into 1.5 ml microcentrifuge tubes. The aliquots were snap frozen in liquid nitrogen and stored at −80° C. until use. Butylated Hydroxytoluene (BHT) was used as the reference standard and was prepared as a 1 mM solution in methanol. A standard curve was prepared from this stock solution in concentrations from 0-1000 uM in varying increments. 2,2-Diphenyl-1-picrylhydrazyl (DPPH) was prepared as a 0.1 mM working solution in methanol. For the assay, a 96-well microtiter was used, and 100 ul DPPH+ 100 ul of sample/standard was added to respective wells. The plate was wrapped in tin foil to protect from the light, and was incubated at room temperature of 60 minutes. Results were read using a plate reader at 517 nm wavelength. DPPH produces violet in methanol solution and fades to shades of yellow color in the presence of antioxidants. Percentage DPPH radical scavenging activity was calculated by the following equation below, whereas A₀ is the absorbance of the control, and A₁ is the absorbance of the extract/standard.

% DPPH radical scavenging activity={(A₀−A₁)/A₀}*100

Using the same supernatant extract prep as above, the Chelating Ferrous Ions assay was performed. EDTA was used as the reference standard was prepared as 1× working solution, from a 10× stock prepared by adding 0.05 g EDTA into 50 ml water, pH at 8.0 to dissolve. Additional reagents prepared were 10× stock Ferrozine (5 mM stock solution) and 10× stock FeCl₂ (2 mM stock solution). An EDTA standard curve was prepared from the stock solution in concentrations of 0-50 mg/L in increments of 10 mg/L. For the experimental setup, a 96-well titer was used and contained a control, a control blank, a sample, and a sample blank. The control was comprised of 100 ul water+50 ul 1× FeCl₂₊₁₀₀ ul 1× Ferrozine. The control blank was comprised of 200 ul water+50 ul 1× FeCl₂. The Sample blank was comprised of 100 ul standard/sample+50 ul 1× FeCl₂₊₁₀₀ ul water. Finally, the sample was comprised of 100 ul standard/sample+50 ul 1× FeCl₂₊₁₀₀ ul 1× Ferrozine. All samples were run in triplicate. The plate was then incubated at room temperature for 5 minutes and read on a plate reader at 562 nm. The respective blanks were subtracted from the control and the sample, and percentage Chelating Activity was calculated by the following equation below, where A₀ is the absorbance of the control, and A₁ is the absorbance of the extract/standard.

% Chelating Activity={(A₀−A₁)/A₀}*100

TABLE 23
Results of Antioxidant Capacity Testing

| Phenotypic | % DPPH Activity | | 75.24% |
|---|---|---|---|
| Activity | % Chelating Activity | | 99.46% |
| Predicted | Primary | Enzymatic | SOD, CAT, GPx |
| Genomic | Antioxidants | | |
| Activity | Secondary | Enzymatic | Glucose-6-phosphate- |
| | Antioxidants | | dehydrogenase |

| | |
|---|---|
| Non-enzymatic Flavonoids | Quercetin |
| | 2,3-dioxygenase |

### Example 28

### Quorum Quenching Capacity

Quorum sensing is a cell's method of communicating, and is a cell density-dependent bacterial response mediated by autoinducer compounds. This communication network controls phenotypic variations including biofilm formation, virulence factor expression, and motility. Quorum quenching is an organism's ability to inhibit or interfere with these communication using chemical or enzymatic means to counteract behaviors regulated by quorum sensing.

Both gram-negative and gram-positive bacterial organisms utilize this type of communication signaling, albeit through different peptide molecules. Gram-negative bacteria predominately utilize Acyl-hemoserine lactones (AHL) molecules such as AI-1, Luxl, or LuxR; while gram-positive bacteria predominately utilize autoinducing peptides (AIP).

Chromobacterium violaceum is a well-studied quorum sensing reporter strain that harbors the LuxIR-type system to detect and respond to changes in cell population density. The hallmark trait of C. violaceum is its production of the purple pigment violacein, which is synthesized during active quorum sensing activity. However, when quorum sensing is inhibited, this reporter strain grows colorless, thus being a good screening tool for quorum quenching activity. In-vitro screening of NRRL No. B-68054 inhibition of C. violaceum quorum sensing communication was tested. Supernatants were prepared from NRRL No. B-68054 for use in these assays. This was completed by inoculation from 10 ul of an overnight culture grown in TSB, and then cultured for 24 hrs in a 250 ml flask with incubation conditions at a temperature of 32° C. and shaking at 180 rpm. The flasks were confirmed for purity, and centrifuged at 4° C., 6000 rpm for 20 minutes to separate the biomass from the supernatant. Supernatant was then filter sterilized through a 0.2 uM filter and aliquoted into 1.5 ml microcentrifuge tubes. The aliquots were snap frozen in liquid nitrogen and stored at −80° C. until use.

C. violaceum strain #12472 was purchased from ATCC and used as the reporter strain for this screening assay. Frozen stock culture was allowed to thaw at room temperature, in a biological safety cabinet (BSC). Additionally, frozen supernatant of NRRL No. B-68054 was allowed to thaw at room temperature, in a BSC. Using a 96-well microtiter plate, test wells were run as 180 ul of TSB, 20 ul NRRL No. B-68054 supernatant, and 2 ul ATCC 12472 C. violaceum strain. The positive control was 200 ul TSB and 2 ul C. violaceum. The negative control was 200 ul TSB. All samples were run in triplicate. The plate was incubated overnight, approximately 16 hrs, at 32° C. The next day, color differences were quantitated by reading the wells on a plate reader at an absorbance of 562 nm. Percentage quorum quenching activity was calculated by the following equation below, where A₀ is the absorbance of the control, and A₁ is the absorbance of the extract/standard.

% Quorum Quenching Activity={(A₀−A₁)/A₀}*100

TABLE 24
Results of Quorum Quenching Activity

| | | |
|---|---|---|
| Phenotypic Activity | % Quorum Quenching | 38.89% |
| | Activity | |

1. A commercial package, feed additive for an animal feed or a human food, additive for the drinking water of an animal or a human, animal feed composition, or a human food composition, said commercial package, feed additive for the animal feed or the human food, the additive for the drinking water of the animal or the human, or the animal feed composition or the human food composition comprising an isolated Bacillus strain selected from the group consisting of Bacillus strain (NRRL No. B-68053), Bacillus strain (NRRL No. B-68054), a strain having all of the identifying characteristics of Bacillus strain (NRRL No. B-68053), a strain having all of the identifying characteristics of Bacillus strain (NRRL No. B-68054), and combinations thereof.

2. The commercial package, the feed additive for the animal feed or human food, the animal feed composition or the human food composition, or the additive for the drinking water of the animal or the human of claim 1, wherein the Bacillus strain inhibits a pathogen selected from the group consisting of E. coli, Salmonella, Staphylococcus, Enterococcus, Campylobacter, Clostridium, Candida, Mucor, Penicillium, and Aspergillus.

3. The commercial package, feed additive for the animal feed or the human food, the additive for the drinking water of the animal or the human, or the animal feed composition or the human food composition of claim 2 further comprising a carrier for the Bacillus strains.

4. The commercial package, feed additive for the animal feed or the human food, the additive for the drinking water of the animal or the human, or the animal feed composition or the human food composition of claim 3 wherein the carrier is selected from the group consisting of a bran, rice hulls, a salt, mineral oil, a dextrin, whey, sugar, limestone, dried starch, sodium silico aluminate, vegetable oil, and combinations thereof.

5. The commercial package, feed additive for the animal feed or the human food, the additive for the drinking water of the animal or the human, or the animal feed composition or the human food composition of claim 2 further comprising a binder.

6. The commercial package, feed additive for the animal feed or the human food, the additive for the drinking water of the animal or the human, or the animal feed composition or the human food composition of claim 5 wherein the binder is selected from the group consisting of clay, yeast cell wall components, aluminum silicate, glucan, and combinations thereof.

7. The commercial package, feed additive for the animal feed or the human food, the additive for the drinking water of the animal or the human, or the animal feed composition or the human food composition of claim 1 in the form of a dietary nutrient composition.

8. The commercial package, feed additive for the animal feed or the human food, the additive for the drinking water of the animal or the human, or the animal feed composition or the human food composition of claim 1 further comprising an exogenously added nutrient component selected from the group consisting of a vitamin, an antibiotic, an enzyme, a water-soluble or water-insoluble monosaccharide, disaccharide, or polysaccharide, a fat, phosphorous, sodium bicarbonate, limestone, calcium, sodium, sulfur, magnesium, potassium, copper, iron, manganese, zinc, fish oil, raw seed, an antioxidant, and a starch.

9. A method of feeding an animal, the method comprising the step of administering to the animal a feed composition or drinking water comprising an effective amount of an additive comprising an isolated Bacillus strain selected from the group consisting of Bacillus strain (NRRL No. B-68053), Bacillus strain (NRRL No. B-68054), a strain having all of the identifying characteristics of Bacillus strain (NRRL No. B-68053), a strain having all of the identifying characteristics of Bacillus strain (NRRL No. B-68054), and combinations thereof.

10. The method of claim 9 wherein at least one of the Bacillus strains has antimicrobial activity.

11. The method of claim 10 wherein the antimicrobial activity is against a microbe selected from the group consisting of E. coli, Salmonella, Staphylococcus, Enterococcus, Clostridia, Campylobacter, Candida, Mucor, Penicillium, Aspergillus, and combinations thereof.

12. The method of claim 9 wherein the strain administered is Bacillus strain (NRRL No. B-68053).

13. The method of claim 9 wherein the strain administered is Bacillus strain (NRRL No. B-68054).

14. A method of improving the health of a human, the method comprising the step of administering to the human a probiotic composition comprising an effective amount of an isolated Bacillus strain selected from the group consisting of Bacillus strain BC1 (NRRL No. B-67744), Bacillus strain (NRRL No. B-68053), Bacillus strain (NRRL No. B-68054), a strain having all of the identifying characteristics of Bacillus strain BC1 (NRRL No. B-67744), a strain having all of the identifying characteristics of Bacillus strain (NRRL No. B-68053), a strain having all of the identifying characteristics of Bacillus strain (NRRL No. B-68054), and combinations thereof.

15. The method of claim 14 wherein the improvement in health is an improvement in gastrointestinal health.

16. The method of claim 14 wherein the Bacillus strain does not cause hemolysis.

17. The method of claim 14 wherein the Bacillus strain does not cause cytotoxicity.

18. The method of claim 14 wherein the Bacillus strain does not produce a toxin selected from the group consisting of hemolysin BL subunit A (hb1A), hemolysin BL subunit C (hb1C), hemolysin BL subunit D (hb1D), nonhemolytic enterotoxin subunit A (nheA), nonhemolytic enterotoxin subunit B (nheB), nonhemolytic enterotoxin subunit C (nheC), emetic toxin, enterotoxin FM (entFM), enterotoxin T (bceT), and cytotoxin K (cytK).

19. The method of claim 14 wherein the Bacillus strain produces an anti-inflammatory biomarker selected from the group consisting of SOCS1, TOLLIP, IL-10, and CXCL12.

20. The method of claim 14 wherein the Bacillus strain has antioxidant activity.

21. The method claim 14 wherein the Bacillus strain has quorum quenching activity.


SEQUENCE LISTING (22 sequences)

SEQ ID NO: 1 - DNA, 25 bp, Synthetic construct
  source 1..25: mol_type = other DNA
attaatacaggggatggagaaactt

SEQ ID NO: 2 - DNA, 27 bp, synthetic construct
  source 1..27: mol_type = other DNA
tgatcctaatacttcttctagacgctt

SEQ ID NO: 3 - DNA, 26 bp, synthetic construct
  source 1..26: mol_type = other DNA
cctatcaatactctcgcaacaccaat

SEQ ID NO: 4 - DNA, 27 bp, synthetic construct
  source 1..27: mol_type = other DNA
ttttcttgattcgtcatagccatttct

SEQ ID NO: 5 - DNA, 30 bp, synthetic construct
  source 1..30: mol_type = other DNA
agatgctacaagacttcaaagggaaactat

SEQ ID NO: 6 - DNA, 27 bp, synthetic construct
  source 1..27: mol_type = other DNA
tgattagcacgatctgctttcatactt

SEQ ID NO: 7 - DNA, 24 bp, synthetic construct
  source 1..24: mol_type = other DNA
agcttggagcggagcagactatgt

SEQ ID NO: 8 - DNA, 24 bp, synthetic construct
  source 1..24: mol_type = other DNA
gtatttctttcccgcttgcctttt

SEQ ID NO: 9 - DNA, 26 bp, synthetic construct
  source 1..26: mol_type = other DNA
attacagggttattggttacagcagt

SEQ ID NO: 10 - DNA, 28 bp, synthetic construct
  source 1..28: mol_type = other DNA
aatcttgctccatactctcttggatgct

SEQ ID NO: 11 - DNA, 20 bp, synthetic construct
  source 1..20: mol_type = other DNA
gtgcagcagctgtaggcggt

SEQ ID NO: 12 - DNA, 26 bp, synthetic construct
  source 1..26: mol_type = other DNA
atgtttttccagctatctttcgcaat

SEQ ID NO: 13 - DNA, 29 bp, synthetic construct
  source 1..29: mol_type = other DNA
gcggatattgtaaagaatcaaaatgaggt

SEQ ID NO: 14 - DNA, 29 bp, synthetic construct
  source 1..29: mol_type = other DNA
tttccagctatctttcgctgtatgtaaat

SEQ ID NO: 15 - DNA, 25 bp, synthetic construct
  source 1..25: mol_type = other DNA
caaagacttcgtaacaaaaggtggt

SEQ ID NO: 16 - DNA, 25 bp, synthetic construct
  source 1..25: mol_type = other DNA
tgtttactccgccttttacaaactt

SEQ ID NO: 17 - DNA, 24 bp, synthetic construct
  source 1..24: mol_type = other DNA
agcttggagcggagcagactatgt

SEQ ID NO: 18 - DNA, 24 bp, synthetic construct
  source 1..24: mol_type = other DNA
gtatttctttcccgcttgcctttt

SEQ ID NO: 19 - DNA, 24 bp, synthetic construct
  source 1..24: mol_type = other DNA
atcgggcaaaatgcaaaaacacat

SEQ ID NO: 20 - DNA, 23 bp, synthetic construct
  source 1..23: mol_type = other DNA
acccagtttgcagttccgaatgt

SEQ ID NO: 21 - DNA, 24 bp, synthetic construct
  source 1..24: mol_type = other DNA
atcggtcaaaatgcaaaaacacat

SEQ ID NO: 22 - DNA, 23 bp, synthetic construct
  source 1..23: mol_type = other DNA
acccagttaccagttccgaatgt